SEQUENCE LISTING (46 sequences)

SEQ ID NO: 1 - DNA, 50 bp, Artificial Sequence
  Synthetic Oligonucleotide
aatttaatacgactcactatagggagagttcaatctgtcaagcagcagca

SEQ ID NO: 2 - DNA, 23 bp, Artificial Sequence
  Synthetic Oligonucleotide
gttcaatctgtcaagcagcagca

SEQ ID NO: 3 - DNA, 49 bp, Artificial Sequence
  Synthetic Oligonucleotide
aatttaatacgactcactatagggagagttgttggcctttaccagacat

SEQ ID NO: 4 - DNA, 22 bp, Artificial Sequence
  Synthetic Oligonucleotide
gttgttggcctttaccagacat

SEQ ID NO: 5 - DNA, 20 bp, Artificial Sequence
  Synthetic Oligonucleotide
gaacttctcctgctagaatg

SEQ ID NO: 6 - DNA, 22 bp, Artificial Sequence
  Synthetic Oligonucleotide
gtaggggaacttctcctgctag

SEQ ID NO: 7 - DNA, 27 bp, Artificial Sequence
  Synthetic Oligonucleotide
aatttaatacgactcactatagggaga

SEQ ID NO: 8 - DNA, 53 bp, Artificial Sequence
  Synthetic Oligonucleotide
cucugcucccuucugcguagtttaaaaaaaaaaaaaaaaaaaaaaaaaaaaaa

SEQ ID NO: 9 - DNA, 20 bp, Artificial Sequence
  Synthetic Oligonucleotide
cucugcucccuucugcguag

SEQ ID NO: 10 - DNA, 53 bp, Artificial Sequence
  Synthetic Oligonucleotide
gaugaggaacgagaagaggctttaaaaaaaaaaaaaaaaaaaaaaaaaaaaaa

SEQ ID NO: 11 - DNA, 20 bp, Artificial Sequence
  Synthetic Oligonucleotide
gaugaggaacgagaagaggc

SEQ ID NO: 12 - DNA, 33 bp, Artificial Sequence
  Synthetic Oligonucleotide
tttaaaaaaaaaaaaaaaaaaaaaaaaaaaaaa

SEQ ID NO: 13 - DNA, 18 bp, Artificial Sequence
  Synthetic Oligonucleotide
uggcggugaugcugcuct

SEQ ID NO: 14 - DNA, 24 bp, Artificial Sequence
  Synthetic Oligonucleotide
ccacaagcuuagaagauagagagg

SEQ ID NO: 15 - DNA, 16 bp, Artificial Sequence
  Synthetic Oligonucleotide
ctatcttctaagcttg

SEQ ID NO: 16 - DNA, 234 bp, Artificial Sequence
  Synthetic Oligonucleotide
cttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaa

SEQ ID NO: 17 - DNA, 73 bp, Artificial Sequence
  Synthetic Oligonucleotide
aatggctggcaatggcggtgatgctgctcttgctttgctgctgcttgacagattgaaccagcttgagagcaaa

SEQ ID NO: 18 - DNA, 69 bp, Artificial Sequence
  Synthetic Oligonucleotide
gctggcaatggcggtgatgctgctcttgctttgctgctgcttgacagattgaaccagcttgagagcaaa

SEQ ID NO: 19 - DNA, 31 bp, Artificial Sequence
  Synthetic Oligonucleotide
aatggctggcaatggcggtgatgctgctctt

SEQ ID NO: 20 - DNA, 27 bp, Artificial Sequence
  Synthetic Oligonucleotide
gctggcaatggcggtgatgctgctctt

SEQ ID NO: 21 - DNA, 21 bp, Artificial Sequence
  Synthetic Oligonucleotide
aatggcggtgatgctgctctt

SEQ ID NO: 22 - DNA, 47 bp, Artificial Sequence
  Synthetic Oligonucleotide
aatttaatacgactcactatagggagagttgttggcctttaccagac

SEQ ID NO: 23 - DNA, 20 bp, Artificial Sequence
  Synthetic Oligonucleotide
gttgttggcctttaccagac

SEQ ID NO: 24 - DNA, 71 bp, Artificial Sequence
  Synthetic Oligonucleotide
gctggcaatggcggtgatgctgctcttgctttgctgctgcttgacagattgaaccagcttgagagcaaaat

SEQ ID NO: 25 - DNA, 75 bp, Artificial Sequence
  Synthetic Oligonucleotide
aatggctggcaatggcggtgatgctgctcttgctttgctgctgcttgacagattgaaccagcttgagagcaaaat

SEQ ID NO: 26 - DNA, 45 bp, Artificial Sequence
  Synthetic Oligonucleotide
aatttaatacgactcactatagggagactttaataacaacattag

SEQ ID NO: 27 - DNA, 18 bp, Artificial Sequence
  Synthetic Oligonucleotide
ctttaataacaacattag

SEQ ID NO: 28 - DNA, 52 bp, Artificial Sequence
  Synthetic Oligonucleotide
aatttaatacgactcactatagggagattgaaattcacagactttaataaca

SEQ ID NO: 29 - DNA, 25 bp, Artificial Sequence
  Synthetic Oligonucleotide
ttgaaattcacagactttaataaca

SEQ ID NO: 30 - DNA, 20 bp, Artificial Sequence
  Synthetic Oligonucleotide
gtactactttagattcgaag

SEQ ID NO: 31 - DNA, 20 bp, Artificial Sequence
  Synthetic Oligonucleotide
gatttttggtactactttag

SEQ ID NO: 32 - DNA, 55 bp, Artificial Sequence
  Synthetic Oligonucleotide
uguuagacuucucaguggaagctttaaaaaaaaaaaaaaaaaaaaaaaaaaaaaa

SEQ ID NO: 33 - DNA, 22 bp, Artificial Sequence
  Synthetic Oligonucleotide
uguuagacuucucaguggaagc

SEQ ID NO: 34 - DNA, 53 bp, Artificial Sequence
  Synthetic Oligonucleotide
gaacucacuuuccauccaactttaaaaaaaaaaaaaaaaaaaaaaaaaaaaaa

SEQ ID NO: 35 - DNA, 20 bp, Artificial Sequence
  Synthetic Oligonucleotide
gaacucacuuuccauccaac

SEQ ID NO: 36 - DNA, 18 bp, Artificial Sequence
  Synthetic Oligonucleotide
gacccagucccuacuuat

SEQ ID NO: 37 - DNA, 16 bp, Artificial Sequence
  Synthetic Oligonucleotide
aagtagggactgggtc

SEQ ID NO: 38 - DNA, 14 bp, Artificial Sequence
  Synthetic Oligonucleotide
agagcagcaucacc

SEQ ID NO: 39 - DNA, 312 bp, Artificial Sequence
  Synthetic Oligonucleotide
tatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgt

SEQ ID NO: 40 - DNA, 31 bp, Artificial Sequence
  Synthetic Oligonucleotide
acccagtccctacttattgttaataacgcta

SEQ ID NO: 41 - DNA, 39 bp, Artificial Sequence
  Synthetic Oligonucleotide
attcgaagacccagtccctacttattgttaataacgcta

SEQ ID NO: 42 - DNA, 38 bp, Artificial Sequence
  Synthetic Oligonucleotide
acccagtccctacttattgttaataacgctactaatgt

SEQ ID NO: 43 - DNA, 46 bp, Artificial Sequence
  Synthetic Oligonucleotide
attcgaagacccagtccctacttattgttaataacgctactaatgt

SEQ ID NO: 44 - DNA, 23 bp, Artificial Sequence
  Synthetic Oligonucleotide
aacttctcctgctagaatggctg

SEQ ID NO: 45 - DNA, 46 bp, Artificial Sequence
  Synthetic Oligonucleotide
aatttaatacgactcactatagggagactttaataacaacattagt

SEQ ID NO: 46 - DNA, 22 bp, Artificial Sequence
  Synthetic Oligonucleotide
ggatttttggtactactttaga

(19) United States
(12) Patent Application Publication
(10) Pub. No.: US 2023/0220499 A1
(43) Pub. Date: Jul. 13, 2023

(54) METHODS AND COMPOSITIONS FOR DETECTING SARS-COV-2 NUCLEIC ACID

(71) Applicant: GRIFOLS DIAGNOSTIC SOLUTIONS INC., Emeryville, CA (US)

(72) Inventors: Kui GAO, Emeryville, CA (US); Anthony JAMES, Emeryville, CA (US); Vanessa BRES, Emeryville, CA (US)

(21) Appl. No.: 17/997,919

(22) PCT Filed: May 7, 2021
(86) PCT No.: PCT/IB2021/053884

§ 371 Date: Nov. 3, 2022

Related U.S. Application Data
(60) Provisional application No. 63/021,326, filed on May 7, 2020.

(51) Int. Cl. C12Q 1/70

(52) U.S. Cl. CPC C12Q 1/701

(57) ABSTRACT

The medical field of COVID-19 diagnosis relates to methods for detecting SARS-CoV-2 nucleic acids in a sample as well as combinations of oligomers for determining the presence or absence of SARS-CoV-2 in a Sample.

### PRIORITY AND CROSS REFERENCE TO RELATED APPLICATIONS

This application is the U.S. National Phase Application under 35 U.S.C. § 371 of International Application No. PCT/IB2021/053884, filed May 7, 2021, designating the U.S. and published as WO2021224873A1 on Nov. 11, 2021, which claims the benefit of Provisional Application No. 63/021,326, filed May 7, 2021. Any and all applications for which a foreign or a domestic priority is claimed is/are identified in the Application Data Sheet filed herewith and is/are hereby incorporated by reference in their entireties under 37 C.F.R. § 1.57.

### FIELD

This application relates to the medical field of COVID-19 diagnosis, and in particular, it relates to methods for detecting SARS-CoV-2 nucleic acids in a sample. This application also relates to combinations of oligomers for determining the presence or absence of SARS-CoV-2 in a sample.

### BACKGROUND

Coronaviruses are a large family of positive-sense single-stranded RNA viruses which may cause illness in animals or humans. In humans, several coronaviruses are known to cause respiratory infections ranging from the common cold to more severe diseases such as Middle East Respiratory Syndrome (MERS) and Severe Acute Respiratory Syndrome (SARS). The most recently discovered coronavirus, SARS-CoV-2, causes the associated coronavirus disease COVID-19. This new virus and disease were unknown before the outbreak began in Wuhan, China, in December 2019.

The most common symptoms of COVID-19 are fever, tiredness, and dry cough. Some patients may have aches and pains, nasal congestion, runny nose, sore throat, or diarrhoea. These symptoms are usually mild and begin gradually. Some people become infected but do not develop any symptoms and do not feel unwell. The disease can spread through respiratory droplets produced when an infected person coughs or sneezes. These droplets land on objects and surfaces around the person. Other people may acquire SARS-CoV-2 by touching these objects or surfaces, then touching their eyes, nose, or mouth.

Person to person spread was subsequently reported worldwide. The World Health Organization (WHO) has designated the pandemic of COVID-19 a Public Health Emergency of International Concern.

Accordingly, there is a need for methods, kits and compositions for detecting the presence or absence of SARS-CoV-2 in a sample with high specificity and sensitivity. Such compositions, kits, and methods would be particularly useful for the diagnosis of COVID-19, for the screening and/or monitoring of the presence of SARS-CoV-2 in a sample, or for monitoring a patient's response to treatment.

### SUMMARY

In some embodiments, a method for detecting SARS-CoV-2 nucleic-acid in a sample includes 1) contacting a sample suspected of containing SARS-CoV-2 nucleic acid with at least two amplification oligomers for amplifying at least one target region of a SARS-CoV-2 target nucleic acid, 2) performing an in vitro nucleis acid amplification reaction and 3) detecting the presence or absence of the amplification product, and indicating the presence or absence of SARS-CoV-2 target nucleic acid in said sample. The two amplification oligomers usually includes a first and a second amplification oligomers for amplifying a first target region of a SARS-CoV-2 nucleic acid, in which the first amplification oligomer includes a first target-hybridizing sequence consisting a sequence selected from the group consisting of SEQ ID NO:2, SEQ ID NO:4 and SEQ ID NO:23; and the second amplification oligomer includes a second target-hybridizing sequence consisting of sequence selected from the group consisting of SEQ ID NO:5, and SEQ ID NO:6. The two amplification oligomers further includes a first and a second amplification oligomers for amplifying a second target region of a SARS-CoV-2 nucleic acid, in which the first amplification oligomer includes a first target-hybridizing sequence consisting of sequence selected from the group consisting of SEQ ID NO:27 and SEQ ID NO:29; and the second amplification oligomer includes a second target-hybridizing sequence consisting of a sequence selected from the group consisting of SEQ ID NO:30 and SEQ ID NO:31. In another embodiments, the first amplification oligomer for amplifying the first and/or the second target region is a promoter primer or promoter provider further comprising a promoter sequence located 5′ to the first target-hybridizing sequence. In another embodiment, the promoter sequence is a T7 promoter sequence.

In some embodiment, the method further includes purifying the target nucleic acid from other components before contacting the sample suspected of containing SARS-CoV-2 nucleic acid with the amplification oligomers. The purifying step includes contacting the sample with at least one capture probe oligomer comprising a target-hybridizing sequence covalently attached to a sequence or moiety that binds to an immobilized probe, in which the target-hybridizing sequence consists of a sequence selected from the group consisting of SEQ ID NO:9, and SEQ ID NO:11 or from the group consisting of SEQ ID NO:33, and SEQ ID NO:35.

In some embodiment, the detecting step includes contacting the in vitro nucleic acid amplification reaction with at least one detection probe oligomer hybridizes to the amplification product under conditions in which the presence or absence of the amplification product is determined, indicating the presence or absence of SARS-CoV-2 in said sample.

In some embodiment, the one detection probe oligomer comprises a target-hybridizing sequence that is from about 14 to about 40 nucleotides in length and hybridizes to a target sequence comprising or consisting of: (a) SEQ ID NO:13, the DNA equivalent of SEQ ID NO:13, the complement of SEQ ID NO:13, the DNA equivalent of the complement of SEQ ID NO:13, or the DNA/RNA chimeric of SEQ ID NO:13, or SEQ ID NO:25, the RNA equivalent of SEQ ID NO:25, the complement of SEQ ID NO:25, the RNA equivalent of the complement of SEQ ID NO:25, or the DNA/RNA chimeric and/or; (b) SEQ ID NO:36, the DNA equivalent of SEQ ID NO: 36, the complement of SEQ ID NO: 36, the DNA equivalent of the complement of SEQ ID NO: 36, or the DNA/RNA chimeric of SEQ ID NO: 36, or SEQ ID NO:43, the RNA equivalent of SEQ ID NO:43, the complement of SEQ ID NO:43, the RNA equivalent of the complement of SEQ ID NO:43, or the DNA/RNA chimeric.

In some embodiment, the first and second target-hybridizing sequences of the first and second amplification oligomers for amplifying the first target region and/or the second target region, respectively comprise or consist of the nucleotide sequences of: (i) for amplifying the first target region consisting of: (a) SEQ ID NO:2, SEQ ID NO:4 or SEQ ID NO:23 and SEQ ID NO:5; or (b) SEQ ID NO:2 SEQ ID NO:4 or SEQ ID NO:23 and SEQ ID NO:6; or (c) SEQ ID NO:2 and SEQ ID NO:5 or SEQ ID NO:6; or (d) SEQ ID NO:4 and SEQ ID NO:5 or SEQ ID NO:6; or (e) SEQ ID NO:23 and SEQ ID NO:5 or SEQ ID NO:6; or (f) SEQ ID NO:2 and SEQ ID NO:5; or (g) SEQ ID NO:2 and SEQ ID NO:6; or (h) SEQ ID NO:4 and SEQ ID NO:5; or (i) SEQ ID NO:4 and SEQ ID NO:6, or (j) SEQ ID NO:23 and SEQ ID NO:5; or (k) SEQ ID NO:23 and SEQ ID NO:6; and (ii) for amplifying the second target region consisting of: (a) SEQ ID NO:27 or SEQ ID NO:29 and SEQ ID NO:30; or (b) SEQ ID NO:27 or SEQ ID NO:29 and SEQ ID NO:31; or (c) SEQ ID NO:27 and SEQ ID NO:30 or SEQ ID NO:31; or (d) SEQ ID NO:29 and SEQ ID NO:30 or SEQ ID NO:31; or (e) SEQ ID NO:27 and SEQ ID NO:30; or (f) SEQ ID NO:27 and SEQ ID NO:31; or (g) SEQ ID NO:29 and SEQ ID NO:30; or (h) SEQ ID NO:29 and SEQ ID NO:31.

In some embodiment, the two amplification oligomers for amplifying the first target region and/or the second target region comprise redundant first amplification oligomers and/or redundant second amplification oligomers. In some embodiment, the first and second target-hybridizing sequences of the redundant first and/or second amplification oligomers for amplifying the first target region and/or the second target region, respectively comprise or consist of the nucleotide sequences of: (i) for amplifying the first target region consisting of: (a) SEQ ID NO:2 and SEQ ID NO:4 and SEQ ID NO:5; or (b) SEQ ID NO:2 and SEQ ID NO:4 and SEQ ID NO:6; or (c) SEQ ID NO:2 and SEQ ID NO:5 and SEQ ID NO:6; or (d) SEQ ID NO:4 and SEQ ID NO:5 and SEQ ID NO:6, or (e) SEQ ID NO:2 and SEQ ID NO:4 and SEQ ID NO:5 and SEQ ID NO:6, or (f) SEQ ID NO:2 and SEQ ID NO:23 and SEQ ID NO:5; or (g) SEQ ID NO:2 and SEQ ID NO:23 and SEQ ID NO:6; or; (h) SEQ ID NO:23 and SEQ ID NO:5 and SEQ ID NO:6, or (i) SEQ ID NO:2 and SEQ ID NO:23 and SEQ ID NO:5 and SEQ ID NO:6, or (j) SEQ ID NO:4 and SEQ ID NO:23 and SEQ ID NO:5; or (k) SEQ ID NO:4 and SEQ ID NO:23 and SEQ ID NO:6; or; (I) SEQ ID NO:4 and SEQ ID NO:23 and SEQ ID NO:5 and SEQ ID NO:6; and (ii) for amplifying the second target region consisting of: (a) SEQ ID NO:27 and SEQ ID NO:29 and SEQ ID NO:30; or (b) SEQ ID NO:27 and SEQ ID NO:29 and SEQ ID NO:31; or (c) SEQ ID NO:27 and SEQ ID NO:30 and SEQ ID NO:31; or (d) SEQ ID NO:29 and SEQ ID NO:30 and SEQ ID NO:31; or (e) SEQ ID NO:27 and SEQ ID NO:29 and SEQ ID NO:30 and SEQ ID NO:31.

In some embodiment, the redundant first and/or second amplification oligomers for amplifying the first and the second target regions respectively comprise or consist of the nucleotide sequences of: (a) SEQ ID NO:2, SEQ ID NO:4, SEQ ID NO:5, SEQ ID NO:6, SEQ ID NO:27, SEQ ID NO:29, SEQ ID NO:30 and SEQ ID NO:31, or (b) SEQ ID NO:2, SEQ ID NO:23, SEQ ID NO:5. SEQ ID NO:6. SEQ ID N:27, SEQ ID NO:29, SEQ ID NO:30 and SEQ ID NO:31, or (c) SEQ ID N:4, SEQ ID NO:23, SEQ ID NO:5, SEQ ID NO:6, SEQ ID NO:27, SEQ ID NO:29, SEQ ID NO:30 and SEQ ID NO:31.

In some embodiment, the two amplification oligomers for amplifying the first and/or the second target region comprise redundant first amplification oligomers and/or redundant second amplification oligomers and/or redundant detection probe oligomers.

In some embodiment, the detection probe oligomer consists of a label selected from the group consisting of a chemiluminescent label, a fluorescent label, quencher and a combination of one or more label. In some embodiment, the amplification reaction is an isothermal amplification reaction, specifically is a transcription-mediated amplification (TMA) reaction. In some embodiment, the detection process is a hybridization protection assay (HPA). In some embodiment, the sample is a clinical sample, a blood sample, a plasma sample, or a serum sample.

In another embodiment, a composition consists of a detection probe oligomer for specifically detecting a SARS-CoV-2 target nucleic acid in a sample, where the detection probe oligomer comprising a target-hybridizing sequence that is from about 14 to about 40 nucleotides in length and is configured to specifically hybridize to a target sequence comprising or consisting of SEQ ID NO:13 or SEQ ID NO: 36, the DNA equivalent of SEQ ID NO:13 or SEQ ID NO: 36, the complement of SEQ ID NO:13 or SEQ ID NO: 36, the DNA equivalent of the complement of SEQ ID NO:13 or SEQ ID NO: 36, or the DNA/RNA chimeric of SEQ ID NO:13 or SEQ ID NO: 36, or SEQ ID NO:25 or SEQ ID NO: 43, the RNA equivalent of SEQ ID NO:25 or SEQ ID NO: 43, the complement of SEQ ID NO:25 or SEQ ID NO: 43, the RNA equivalent of the complement of SEQ ID NO:25 or SEQ ID NO: 43, or the DNA/RNA chimeric. The detection probe target-hybridizing sequence of the composition is consisting of a sequence of SEQ ID NO:17 or SEQ ID NO: 41, and includes at least the sequence of SEQ ID NO:13 or SEQ ID NO: 36. The detection probe oligomer in the composition consists of a label selected from the group consisting of a chemiluminescent label, a fluorescent label, quencher and a combination of one or more labels.

### DETAILED DESCRIPTION

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

The term “sample” includes any specimen that may contain, or is suspected of containing, SARS-CoV-2 nucleic acid or components thereof, such as nucleic acids or fragments of SARS-CoV-2 nucleic acids. The sample may be an isolated sample. Samples include “biological samples” which include any tissue or material derived from a living or dead human that may contain the SARS-CoV-2 or components thereof (e.g., a target nucleic acid derived therefrom). Samples also include “environmental samples” and sampling devices (e.g., swabs), which are brought into contact with biological or environmental samples.

“Biological samples” include body fluids such as urine, blood, plasma, serum, peripheral blood, red blood cells, lymph node, gastrointestinal tissue, faecal matter, cerebrospinal fluid (CSF), semen, sputum, saliva, or other body fluids or materials as well as solid tissue. Biological samples also include a cell (such as cell lines, cells isolated from tissue whether or not the isolated cells are cultured after isolation from tissue, fixed cells such as cells fixed for histological and/or immunohistochemical analysis), tissue (such as biopsy material), or fluid obtained from a mammal, including from the upper respiratory tissues (such as nasopharyngeal wash, nasopharyngeal aspirate, nasopharyngeal swab, and oropharyngeal swab), from the lower respiratory tissues (such as bronchiolar lavage, tracheal aspirate, pleural tap, sputum), and tissue from any organ such as, without limitation, lung, heart, spleen, liver, brain, kidney, and adrenal glands. Nucleic acids (e.g., DNA and RNA) isolated from a cell and/or tissue, and the like are also included.

“Environmental samples” include environmental material such as surface matter, soil, water, and industrial materials, as well as material obtained from food and dairy processing instruments, apparatus, equipment, disposable, and non-disposable items.

The sample may be treated to physically or mechanically disrupt tissue or cell structure, thus releasing intracellular components into a solution which may further contain enzymes, buffers, salts, detergents and the like, which are used to prepare, using standard methods, a biological sample for analysis. Also, samples may include processed samples, such as those obtained from passing samples over or through a filtering device, or following centrifugation, or by adherence to a medium, matrix, or support.

The term “nucleic acid” refers to a multimeric compound comprising two or more covalently bonded nucleosides or nucleoside analogues having nitrogenous heterocyclic bases, or base analogues, where the nucleosides are linked together by phosphodiester bonds or other linkages to form a polynucleotide. Nucleic acids include RNA, DNA, or chimeric DNA-RNA polymers or oligonucleotides, and analogues thereof. A nucleic acid “backbone” may be made up of a variety of linkages, including one or more of sugar-phosphodiester linkages, peptide-nucleic acid bonds (in “peptide nucleic acids” or PNAs, see WO95/32305), phosphorothioate linkages, methylphosphonate linkages, or combinations thereof. Sugar moieties of the nucleic acid may be either ribose or deoxyribose, or similar compounds having known substitutions, e.g., 2′ methoxy substitutions and 2′ halide substitutions (e.g., 2′-F). Nitrogenous bases may be conventional bases (A, G, C, T, U), analogs thereof (e.g., inosine, 5-methylisocytosine, isoguanine; The Biochemistry of the Nucleic Acids 5-36, Adams et al, ed., 11th ed., 1992, BioTechniques (2007) 43: 617-24), which include derivatives of purine or pyrimidine bases (e.g., N4-methyl deoxygaunosine, deaza- or aza-purines, deaza- or aza-pyrimidines, pyrimidine bases having substituent groups at the 5 or 6 position, purine bases having an altered or replacement substituent at the 2, 6 and/or 8 position, such as 2-amino-6-methylaminopurine, 06-methylguanine, 4-thio-pyrimidines, 4-amino-pyrimidines, 4-dimethylhydrazine-pyrimidines, and 04-alkyl-pyrimidines, and pyrazolo-compounds, such as unsubstituted or 3-substituted pyrazolo[3,4-d]pyrimidine; U.S. Pat. Nos. 5,378,825, 6,949,367 and PCT No. WO 93/13121). Nucleic acids may include “abasic” residues in which the backbone does not include a nitrogenous base for one or more residues (U.S. Pat. No. 5,585,481). A nucleic acid may comprise only conventional sugars, bases, and linkages as found in RNA and DNA, or may include conventional components and substitutions (e.g., conventional bases linked by a 2′ methoxy backbone, or a nucleic acid including a mixture of conventional bases and one or more base analogues). Nucleic acids may include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA) (Biochemistry (2004) 43: 13233-41). Nucleic acids may include modified bases to alter the function or behaviour of the nucleic acid, e.g., addition of a 3′-terminal dideoxynucleotide to block additional nucleotides from being added to the nucleic acid. Synthetic methods for making nucleic acids in vitro are well-known in the art although nucleic acids may be purified from natural sources using routine techniques.

The term “polynucleotide,” as used herein, denotes a nucleic acid chain. Throughout this application, nucleic acids are designated by the 5′-terminus to the 3′-terminus. Synthetic nucleic acids, e.g., DNA, RNA, DNA/RNA chimerics (including when non-natural nucleotides or analogues are included therein), are typically synthesized “3′-to-5′,” i.e., by the addition of nucleotides to the 5′-terminus of a growing nucleic acid.

A “nucleotide,” as used herein, is a subunit of a nucleic acid consisting of a phosphate group, a 5-carbon sugar and a nitrogenous base. The 5-carbon sugar found in RNA is ribose. In DNA, the 5-carbon sugar is 2′-deoxyribose. The term also includes analogs of such subunits, such as a methoxy group at the 2′ position of the ribose (2′-O-Me).

A “nucleic-acid-based detection assay,” as used herein, is an assay for the detection of a target sequence within a target nucleic acid and utilizing one more oligonucleotides that specifically hybridize to the target sequence.

In certain embodiments, a nucleic-acid-based detection assay is an “amplification-based assay,” i.e., an assay that utilizes one or more steps for amplifying a nucleic acid target sequence. Various amplification methods for use in detection assays are known in the art, several of which are summarized further herein. For the sake of clarity, an amplification-based assay may include one or more steps that do not amplify a target sequence, such as, for example, steps used in non-amplification-based assay methods (e.g., a hybridization assay or a cleavage-based assay).

In other embodiments, a nucleic-acid-based detection assay is a “non-amplification-based assay,” i.e., an assay that does not rely on any step for amplifying a nucleic acid target sequence. For the sake of clarity, a nucleic-acid-based detection assay that includes a reaction for extension of a primer in the absence of any corresponding downstream amplification oligomer (e.g., extension of a primer by a reverse transcriptase to generate an RNA:DNA duplex followed by an RNase digestion of the RNA, resulting in a single-stranded cDNA complementary to an RNA target but without generating copies of the cDNA) is understood to be a non-amplification-based assay.

A “target nucleic acid,” as used herein, is a nucleic acid comprising a target sequence to be detected. Target nucleic acids may be DNA or RNA as described herein, and may be either single-stranded or double-stranded. The target nucleic acid may include other sequences besides the target sequence.

By “isolated” it is meant that a sample containing a target nucleic acid is taken from its natural milieu, but the term does not connote any degree of purification.

The term “target sequence,” as used herein, refers to the particular nucleotide sequence of a target nucleic acid that is to be detected. The “target sequence” includes the complexing sequences to which oligonucleotides (e.g., probe oligonucleotide, priming oligonucleotides and/or promoter oligonucleotides) complex during a detection process (e.g., an amplification-based detection assay such as, for example, TMA or PCR, or a non-amplification-based detection assay such as, for example, a cleavage-based assay). Where the target nucleic acid is originally single-stranded, the term “target sequence” will also refer to the sequence complementary to the “target sequence” as present in the target nucleic acid. Where the target nucleic acid is originally double-stranded, the term “target sequence” refers to both the sense (+) and antisense (−) strands. In choosing a target sequence, the skilled artisan will understand that a “unique” sequence should be chosen so as to distinguish between unrelated or closely related target nucleic acids.

“Target-hybridizing sequence” is used herein to refer to the portion of an oligomer that is configured to hybridize with a target nucleic acid sequence. Preferably, the target-hybridizing sequences are configured to specifically hybridize with a target nucleic acid sequence. Target-hybridizing sequences may be 100% complementary to the portion of the target sequence to which they are configured to hybridize, but not necessarily. Target-hybridizing sequences may also include inserted, deleted and/or substituted nucleotide residues relative to a target sequence. Less than 100% complementarity of a target-hybridizing sequence to a target sequence may arise, for example, when the target nucleic acid is a plurality strains within a species, such as would be the case for an oligomer configured to hybridize to various genotypes of SARS-CoV-2. It is understood that other reasons exist for configuring a target-hybridizing sequence to have less than 100% complementarity to a target nucleic acid.

The term “targets a sequence,” as used herein in reference to a region of SARS-CoV-2 nucleic acid, refers to a process whereby an oligonucleotide hybridizes to the target sequence in a manner that allows for detection as described herein. In some embodiments, the oligonucleotide is complementary with the targeted SARS-CoV-2 nucleic acid sequence and contains no mismatches. In other embodiments, the oligonucleotide is complementary but contains 1, 2, 3, 4, or 5 mismatches with the targeted SARS-CoV-2 nucleic acid sequence. Preferably, the oligonucleotide that hybridizes to the target nucleic acid sequence includes at least 10 to as many as 50 nucleotides complementary to the target sequence. It is understood that at least 10 and as many as 50 is an inclusive range such that 10, 50 and each whole number there between are included. Preferably, the oligomer specifically hybridizes to the target sequence.

The term “configured to” denotes an actual arrangement of the polynucleotide sequence configuration of a referenced oligonucleotide target-hybridizing sequence. For example, oligonucleotides that are configured to specifically hybridize to a target sequence have a polynucleotide sequence that specifically hybridizes to the referenced sequence under stringent hybridization conditions.

The term “configured to specifically hybridize to” as used herein means that the target-hybridizing region of an oligonucleotide is designed to have a polynucleotide sequence that could target a sequence of the referenced SARS-CoV-2 target region. Such an oligonucleotide is not limited to targeting that sequence only, but is rather useful as a composition, in a kit or in a method for targeting a SARS-CoV-2 target nucleic acid. The oligonucleotide is designed to function as a component of an assay for detection of SARS-CoV-2 from a sample, and therefore is designed to target SARS-CoV-2 in the presence of other nucleic acids commonly found in testing samples. “Specifically hybridize to” does not mean exclusively hybridize to, as some small level of hybridization to non-target nucleic acids may occur, as is understood in the art. Rather, “specifically hybridize to” means that the oligonucleotide is configured to function in an assay to primarily hybridize the target so that an accurate detection of target nucleic acid in a sample can be determined. The term “configured to” denotes an actual arrangement of the polynucleotide sequence configuration of the oligonucleotide target-hybridizing sequence.

The term “fragment,” as used herein in reference to a SARS-CoV-2 targeted nucleic acid, refers to a piece of contiguous nucleic acid.

The term “region,” as used herein, refers to a portion of a nucleic acid wherein said portion is smaller than the entire nucleic acid. For example, when the nucleic acid in reference is an oligonucleotide promoter primer, the term “region” may be used refer to the smaller promoter portion of the entire oligonucleotide. As a non-limiting example, when the nucleic acid in reference is an amplicon, the term region may be used to refer to the smaller nucleotide sequence identified for hybridization by the target-hybridizing sequence of a probe.

The interchangeable terms “oligomer”, “oligo”, and “oligonucleotide” refer to a nucleic acid (including RNA, DNA, or chimeric DNA-RNA polymers or oligonucleotides, and analogs thereof) having generally less than 1,000 nucleotide (nt) residues, including polymers in a range having a lower limit of about 5 nt residues and an upper limit of about 500 to 900 nt residues. In some embodiments, oligonucleotides are in a size range having a lower limit of about 12 to 15 nt and an upper limit of about 50 to 600 nt, and other embodiments are in a range having a lower limit of about 15 to 20 nt and an upper limit of about 22 to 100 nt. Oligonucleotides may be purified from naturally occurring sources or may be synthesized using any of a variety of well-known enzymatic or chemical methods. The term oligonucleotide does not denote any particular function to the reagent; rather, it is used generically to cover all such reagents described herein. An oligonucleotide may serve various different functions. For example, it may function as a primer if it is specific for and capable of hybridizing to a complementary strand and can further be extended in the presence of a nucleic acid polymerase; it may function as a primer and provide a promoter if it contains a sequence recognized by an RNA polymerase and allows for transcription (e.g., a T7 Primer); and it may function to detect a target nucleic acid if it is capable of hybridizing to the target nucleic acid, or an amplicon thereof, and further provides a detectible moiety (e.g., an acridinium-ester compound).

As used herein, an oligonucleotide can “substantially correspond to” a specified reference nucleic acid sequence, which means that the oligonucleotide is sufficiently similar to the reference nucleic acid sequence such that the oligonucleotide has similar hybridization properties to the reference nucleic acid sequence in that it would hybridize with the same target nucleic acid sequence under stringent hybridization conditions. One skilled in the art will understand that “substantially corresponding oligonucleotides” can vary from a reference sequence and still hybridize to the same target nucleic acid sequence. It is also understood that a first nucleic acid corresponding to a second nucleic acid includes the RNA or DNA equivalent thereof as well as DNA/RNA chimerics thereof, and includes the complements thereof, unless the context clearly dictates otherwise. This variation from the nucleic acid may be stated in terms of a percentage of identical bases within the sequence or the percentage of perfectly complementary bases between the probe or primer and its target sequence. Thus, in certain embodiments, an oligonucleotide “substantially corresponds” to a reference nucleic acid sequence if these percentages of base identity or complementarity are from 100% to about 80%. In preferred embodiments, the percentage is from 100% to about 85%. In more preferred embodiments, this percentage is from 100% to about 90%; in other preferred embodiments, this percentage is from 100% to about 95%. Similarly, a region of a nucleic acid or amplified nucleic acid can be referred to herein as corresponding to a reference nucleic acid sequence. One skilled in the art will understand the various modifications to the hybridization conditions that might be required at various percentages of complementarity to allow hybridization to a specific target sequence without causing an unacceptable level of non-specific hybridization.

As used herein, the phrase “the RNA equivalent, the complement, the RNA equivalent of the complement or a DNA/RNA chimeric thereof,” with reference to a DNA sequence, includes (in addition to the referenced DNA sequence) the RNA equivalent of the referenced DNA sequence, the complement of the DNA sequence, the RNA equivalent of the complement of the referenced DNA sequence, the DNA/RNA chimeric of the referenced DNA sequence, and a DNA/RNA chimeric of the complement of the referenced DNA sequence. Similarly, the phrase “the DNA equivalent, the complement, the DNA equivalent of the complement or a DNA/RNA chimeric thereof,” with reference to an RNA sequence, includes (in addition to the referenced RNA sequence) the DNA equivalent of the referenced RNA sequence, the complement of the RNA sequence, the DNA equivalent of the complement of the referenced RNA sequence, the DNA/RNA chimeric of the referenced RNA sequence, and a DNA/RNA chimeric of the complement of the referenced RNA sequence.

As used herein, a “blocking moiety” is a substance used to “block” the 3′-terminus of an oligonucleotide or other nucleic acid so that it cannot be efficiently extended by a nucleic acid polymerase. Oligomers not intended for extension by a nucleic acid polymerase may include a blocker group that replaces the 3′ OH to prevent enzyme-mediated extension of the oligomer in an amplification reaction. For example, blocked amplification oligomers and/or detection probes present during amplification may not have functional 3′ OH and instead include one or more blocking groups located at or near the 3′ end. In some embodiments a blocking group near the 3′ end and may be within five residues of the 3′ end and is sufficiently large to limit binding of a polymerase to the oligomer. In other embodiments a blocking group is covalently attached to the 3′ terminus. Many different chemical groups may be used to block the 3′ end, e.g., alkyl groups, non-nucleotide linkers, alkane-diol dideoxynucleotide residues, and cordycepin.

An “amplification oligomer” is an oligomer, at least the 3′-end of which is complementary to a target nucleic acid, and which hybridizes to a target nucleic acid, or its complement, and participates in a nucleic acid amplification reaction. An example of an amplification oligomer is a “primer” that hybridizes to a target nucleic acid and contains a 3′ OH end that is extended by a polymerase in an amplification process. Another example of an amplification oligomer is an oligomer that is not extended by a polymerase (e.g., because it has a 3′ blocked end) but participates in or facilitates amplification. For example, the 5′ region of an amplification oligonucleotide—such as a first amplification oligomer as described herein—may include a promoter sequence that is non-complementary to the target nucleic acid (which may be referred to as a “promoter primer” or “promoter provider”). Those skilled in the art will understand that an amplification oligomer that functions as a primer may be modified to include a 5′ promoter sequence, and thus function as a promoter primer. Incorporating a 3′ blocked end further modifies the promoter primer, which is now capable of hybridizing to a target nucleic acid and providing an upstream promoter sequence that serves to initiate transcription, but does not provide a primer for oligo extension. Such a modified oligo is referred to herein as a “promoter provider” oligomer. Size ranges for amplification oligonucleotides include those that are about 10 to about 70 nt long (not including any promoter sequence or poly-A tails) and contain at least about 10 contiguous bases, or even at least 12 contiguous bases that are complementary to a region of the target nucleic acid sequence (or a complementary strand thereof). The contiguous bases are at least 80%, or at least 90%, or completely complementary to the target sequence to which the amplification oligomer binds. An amplification oligomer may optionally include modified nucleotides or analogs, or additional nucleotides that participate in an amplification reaction but are not complementary to or contained in the target nucleic acid, or template sequence. It is understood that when referring to ranges for the length of an oligonucleotide, amplicon, or other nucleic acid, that the range is inclusive of all whole numbers (e.g., 19-25 contiguous nucleotides in length includes 19, 20, 21, 22, 23, 24 & 25).

As used herein, a “promoter” is a specific nucleic acid sequence that is recognized by a DNA-dependent RNA polymerase (“transcriptase”) as a signal to bind to the nucleic acid and begin the transcription of RNA at a specific site.

As used herein, a “promoter provider” or “provider” refers to an oligonucleotide comprising first and second regions, and which is modified to prevent the initiation of DNA synthesis from its 3′-terminus. The “first region” of a promoter provider oligonucleotide comprises a base sequence which hybridizes to a DNA template, where the hybridizing sequence is situated 3′, but not necessarily adjacent to, a promoter region. The hybridizing portion of a promoter oligonucleotide is typically at least 10 nucleotides in length, and may extend up to 50 or more nucleotides in length. The “second region” comprises a promoter sequence for an RNA polymerase. A promoter oligonucleotide is engineered so that it is incapable of being extended by an RNA- or DNA-dependent DNA polymerase, e.g., reverse transcriptase, preferably comprising a blocking moiety at its 3′-terminus as described above. As referred to herein, a “T7 Provider” is a blocked promoter provider oligonucleotide that provides an oligonucleotide sequence that is recognized by T7 RNA polymerase.

“Amplification” refers to any known procedure for obtaining multiple copies of a target nucleic acid sequence or its complement or fragments thereof. The multiple copies may be referred to as amplicons or amplification products. Known amplification methods include both thermal cycling and isothermal amplification methods. In some embodiments, isothermal amplification methods are preferred. Replicase-mediated amplification, polymerase chain reaction (PCR), ligase chain reaction (LCR), strand-displacement amplification (SDA), and transcription-mediated or transcription-associated amplification are non-limiting examples of nucleic acid amplification methods. Replicase-mediated amplification uses self-replicating RNA molecules, and a replicase such as QB-replicase (e.g., U.S. Pat. No. 4,786,600). PCR amplification uses a DNA polymerase, pairs of primers, and thermal cycling to synthesize multiple copies of two complementary strands of dsDNA or from a cDNA (e.g., U.S. Pat. Nos. 4,683,195, 4,683,202, and 4,800,159). LCR amplification uses four or more different oligonucleotides to amplify a target and its complementary strand by using multiple cycles of hybridization, ligation, and denaturation (e.g., U.S. Pat. Nos. 5,427,930 and 5,516,663). SDA uses a primer that contains a recognition site for a restriction endonuclease and an endonuclease that nicks one strand of a hemimodified DNA duplex that includes the target sequence, whereby amplification occurs in a series of primer extension and strand displacement steps (e.g., U.S. Pat. Nos. 5,422,252; 5,547,861; and 5,648,211). Preferred embodiments use an amplification method suitable for the amplification of RNA target nucleic acids, such as transcription-mediated amplification (TMA) or NASBA, but it will be apparent to persons of ordinary skill in the art that oligomers disclosed herein may be readily used as primers in other amplification methods.

“Transcription-associated amplification,” also referred to herein as “transcription-mediated amplification” (TMA), refers to nucleic acid amplification that uses an RNA polymerase to produce multiple RNA transcripts from a nucleic acid template. These methods generally employ an RNA polymerase, a DNA polymerase, deoxyribonucleoside triphosphates, ribonucleoside triphosphates, and a template complementary oligonucleotide that includes a promoter sequence, and optionally may include one or more other oligonucleotides. TMA methods are embodiments of amplification methods used for amplifying and detecting target sequences as described herein. Variations of transcription-associated amplification are well-known in the art as previously disclosed in detail (e.g., U.S. Pat. Nos. 4,868,105; 5,124,246; 5,130,238; 5,437,990; 5,554,516; and 7,374,885; and PCT Pub. Nos. WO 88/01302, WO 88/10315, and WO 95/03430). The person of ordinary skill in the art will appreciate that the disclosed compositions may be used in amplification methods based on extension of oligomer sequences by a polymerase.

As used herein, the term “real-time TMA” refers to single-primer transcription-mediated amplification (“TMA”) of target nucleic acid that is monitored by real-time detection means.

The term “amplicon,” which is used interchangeably with “amplification product,” refers to the nucleic acid molecule generated during an amplification procedure that is complementary or homologous to a sequence contained within the target sequence. These terms can be used to refer to a single strand amplification product, a double strand amplification product or one of the strands of a double strand amplification product. The complementary or homologous sequence of an amplicon is sometimes referred to herein as a “target-specific sequence.” Amplicons generated using the amplification oligomers of the current invention may comprise non-target specific sequences. Amplicons can be double-stranded or single-stranded and can include DNA, RNA, or both. For example, DNA-dependent RNA polymerase transcribes single-stranded amplicons from double-stranded DNA during transcription-mediated amplification procedures. These single-stranded amplicons are RNA amplicons and can be either strand of a double-stranded complex, depending on how the amplification oligomers are configured. Thus, amplicons can be single-stranded RNA. RNA-dependent DNA polymerases synthesize a DNA strand that is complementary to an RNA template. Thus, amplicons can be double-stranded DNA and RNA hybrids. RNA-dependent DNA polymerases often include RNase activity, or are used in conjunction with an RNase, which degrades the RNA strand. Thus, amplicons can be single stranded DNA. RNA-dependent DNA polymerases and DNA-dependent DNA polymerases synthesize complementary DNA strands from DNA templates. Thus, amplicons can be double-stranded DNA. RNA-dependent RNA polymerases synthesize RNA from an RNA template. Thus, amplicons can be double-stranded RNA. DNA-dependent RNA polymerases synthesize RNA from double-stranded DNA templates, also referred to as transcription. Thus, amplicons can be single stranded RNA. Amplicons and methods for generating amplicons are known to those skilled in the art. For convenience herein, a single strand of RNA or a single strand of DNA may represent an amplicon generated by an amplification oligomer combination of the current invention. Such representation is not meant to limit the amplicon to the representation shown. Skilled artisans in possession of the instant disclosure will use amplification oligomers and polymerase enzymes to generate any of the numerous types of amplicons, all within the spirit and scope of the current invention.

As used herein, a “DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly, or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DNA-dependent DNA polymerase may synthesize a complementary DNA copy from an RNA template. RNA-dependent DNA polymerases typically also have DNA-dependent DNA polymerase activity.

As used herein, a “DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in the 5′-to-3′ direction beginning at a specific position just downstream of the promoter. Examples of transcriptases are the DNA-dependent RNA polymerase from E. coli and bacteriophages T7, T3, and SP6.

As used herein, an “RNA-dependent DNA polymerase” or “reverse transcriptase” (“RT”) is an enzyme that synthesizes a complementary DNA copy from an RNA template. All known reverse transcriptases also have the ability to make a complementary DNA copy from a DNA template; thus, they are both RNA- and DNA-dependent DNA polymerases.

RTs may also have an RNAse H activity. A primer is required to initiate synthesis with both RNA and DNA templates.

As used herein, a “selective RNAse” is an enzyme that degrades the RNA portion of an RNA:DNA duplex but not single-stranded RNA, double-stranded RNA or DNA. An exemplary selective RNAse is RNAse H. Enzymes possessing the same or similar activity as RNAse H may also be used. Selective RNAses may be endonucleases or exonucleases. Most reverse transcriptase enzymes contain an RNAse H activity in addition to their polymerase activities. However, other sources of the RNAse H are available without an associated polymerase activity. The degradation may result in separation of RNA from a RNA:DNA complex. Alternatively, a selective RNAse may simply cut the RNA at various locations such that portions of the RNA melt off or permit enzymes to unwind portions of the RNA. Other enzymes that selectively degrade RNA target sequences or RNA products of the present invention will be readily apparent to those of ordinary skill in the art.

“Probe,” “detection probe,” “detection oligonucleotide,” and “detection probe oligomer” are used interchangeably herein to refer to a nucleic acid oligomer that hybridizes specifically to a target sequence in a nucleic acid, or in an amplified nucleic acid, under conditions that promote hybridization to allow detection of the target sequence or amplified nucleic acid. Detection may either be direct (e.g., a probe hybridized directly to its target sequence) or indirect (e.g., a probe linked to its target via an intermediate molecular structure). Probes may be DNA, RNA, analogues thereof or combinations thereof (e.g., DNA/RNA chimerics) and they may be labeled or unlabeled. Detection probes may further include alternative backbone linkages such as, e.g., 2′-O-methyl linkages. A probe's “target sequence” generally refers to a smaller nucleic acid sequence within a larger nucleic acid sequence that hybridizes specifically to at least a portion of a probe oligomer by standard base pairing. A probe may comprise target-specific sequences and other sequences that contribute to the three-dimensional conformation of the probe (e.g., U.S. Pat. Nos. 5,118,801; 5,312,728; 6,849,412; 6,835,542; 6,534,274; and 6,361,945; and US Pub. No. 20060068417). In a preferred embodiment, the detection probe comprises a 2′ methoxy backbone which can result in a higher signal being obtained.

As used herein, a “label” refers to a moiety or compound joined directly or indirectly to a probe that is detected or leads to a detectable signal. Direct labelling can occur through bonds or interactions that link the label to the probe, including covalent bonds or non-covalent interactions, e.g., hydrogen bonds, hydrophobic and ionic interactions, or formation of chelates or coordination complexes. Indirect labelling can occur through use of a bridging moiety or “linker” such as a binding pair member, an antibody or additional oligomer, which is either directly or indirectly labeled, and which may amplify the detectable signal. Labels include any detectable moiety, such as a radionuclide, ligand (e.g., biotin, avidin), enzyme or enzyme substrate, reactive group, or chromophore (e.g., dye, particle, or bead that imparts detectable color), luminescent compound (e.g., bioluminescent, phosphorescent, or chemiluminescent labels), or fluorophore. Labels may be detectable in a homogeneous assay in which bound labeled probe in a mixture exhibits a detectable change different from that of an unbound labeled probe, e.g., instability or differential degradation properties. A “homogeneous detectable label” can be detected without physically removing bound from unbound forms of the label or labeled probe (e.g., U.S. Pat. Nos. 5,283,174, 5,656,207, and 5,658,737). Labels include chemiluminescent compounds, e.g., acridinium ester (“AE”) compounds that include standard AE and derivatives (e.g., U.S. Pat. Nos. 5,656,207, 5,658,737, and 5,639,604). Synthesis and methods of attaching labels to nucleic acids and detecting labels are well known (e.g., Sambrook et al., Molecular Cloning, A Laboratory Manual, 2nd ed. (Cold Spring Harbor Laboratory Press, Cold Spring Harbor, N.Y., 1989), Chapter 10; U.S. Pat. Nos. 5,658,737, 5,656,207, 5,547,842, 5,283,174, and 4,581,333). More than one label, and more than one type of label, may be present on a particular probe, or detection may use a mixture of probes in which each probe is labeled with a compound that produces a detectable signal (e.g., U.S. Pat. Nos. 6,180,340 and 6,350,579).

“Capture probe,” “capture oligonucleotide,” “target capture oligonucleotide,” and “capture probe oligomer” are used interchangeably herein to refer to a nucleic acid oligomer that specifically hybridizes to a target sequence in a target nucleic acid by standard base pairing and joins to a binding partner on an immobilized probe to capture the target nucleic acid to a support. One example of a capture oligomer includes an oligonucleotide comprising two binding regions: a target hybridizing sequence and an immobilized probe-binding region. A variation of this example, the two regions may be present on two different oligomers joined together by one or more linkers. Another embodiment of a capture oligomer the target hybridizing sequence is a sequence that includes random or non-random poly-GU, poly-GT, or poly-U sequences to bind non-specifically to a target nucleic acid and link it to an immobilized probe on a support (see, e.g., WO 2008/016988). Another example of a capture oligomer comprises two regions, a target hybridizing sequence and a binding pair member that is not a nucleic acid sequence.

As used herein, an “immobilized oligonucleotide,” “immobilized probe” or “immobilized nucleic acid” refers to a nucleic acid binding partner that joins a capture oligomer to a support, directly or indirectly. An immobilized probe joined to a support facilitates separation of a capture probe bound target from unbound material in a sample. One embodiment of an immobilized probe is an oligomer joined to a support that facilitates separation of bound target sequence from unbound material in a sample. Supports may include known materials, such as matrices and particles free in solution, which may be made of nitrocellulose, nylon, glass, polyacrylate, mixed polymers, polystyrene, silane, polypropylene, metal, or other compositions, of which one embodiment is magnetically attractable particles. Supports may be monodisperse magnetic spheres (e.g., uniform size+5%), to which an immobilized probe is joined directly (via covalent linkage, chelation, or ionic interaction), or indirectly (via one or more linkers), where the linkage or interaction between the probe and support is stable during hybridization conditions.

As used herein, a “probe protection” or “probe protection oligomer” are used interchangeably to refer to a nucleic acid oligomer that is substantially complementary to a detection probe oligomer. A probe protection oligomer may be hybridized to a substantially complementary, labeled detection probe oligomer (e.g., a probe labeled with a chemiluminescent compound) to stabilize the labeled probe during storage. Said probe protection oligomers may also be used to adjust assay sensitivity.

As used herein, the term “complementary” means that the nucleotide sequences of similar regions of two single-stranded nucleic acids, or two different regions of the same single-stranded nucleic acid, have a nucleotide base composition that allow the single-stranded regions to hybridize together in a stable double-stranded hydrogen-bonded region under stringent hybridization or amplification conditions. Sequences that hybridize to each other may be completely complementary or partially complementary to the intended target sequence by standard nucleic acid base pairing (e.g., G:C, A:T, or A:U pairing). By “sufficiently complementary” is meant a contiguous sequence that is capable of hybridizing to another sequence by hydrogen bonding between a series of complementary bases, which may be complementary at each position in the sequence by standard base pairing or may contain one or more residues, including abasic residues, that are not complementary. Sufficiently complementary contiguous sequences typically are at least 80%, or at least 90%, complementary to a sequence to which an oligomer is intended to specifically hybridize. Sequences that are “sufficiently complementary” allow stable hybridization of a nucleic acid oligomer with its target sequence under appropriate hybridization conditions, even if the sequences are not completely complementary. When a contiguous sequence of nucleotides of one single-stranded region is able to form a series of “canonical” hydrogen-bonded base pairs with an analogous sequence of nucleotides of the other single-stranded region, such that A is paired with U or T and C is paired with G, the nucleotides sequences are “completely” complementary (see, e.g., Sambrook et al., Molecular Cloning, A Laboratory Manual, 2nd ed. (Cold Spring Harbor Laboratory Press, Cold Spring Harbor, N.Y., 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein). It is understood that ranges for percent identity are inclusive of all whole and partial numbers (e.g., at least 90% includes 90, 91, 93.5, 97.687 and etc.).

By “preferentially hybridize” or “specifically hybridize” is meant that under stringent hybridization assay conditions, probes hybridize to their target sequences, or replicates thereof, to form stable probe:target hybrids, while at the same time formation of stable probe:non-target hybrids is minimized. Thus, a probe hybridizes to a target sequence or replicate thereof to a sufficiently greater extent than to a non-target sequence, to enable one having ordinary skill in the art to accurately detect or quantitate RNA replicates or complementary DNA (cDNA) of the target sequence formed during the amplification. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, N.Y., 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

By “nucleic acid hybrid,” “hybrid,” or “duplex” is meant a nucleic acid structure containing a double-stranded, hydrogen-bonded region wherein each strand is complementary to the other, and wherein the region is sufficiently stable under stringent hybridization conditions to be detected by means including, but not limited to, chemiluminescent or fluorescent light detection, autoradiography, or gel electrophoresis. Such hybrids may comprise RNA:RNA, RNA:DNA, or DNA:DNA duplex molecules.

“Sample preparation” refers to any steps or method that treats a sample for subsequent amplification and/or detection of SARS-CoV-2 nucleic acids present in the sample. Samples may be complex mixtures of components of which the target nucleic acid is a minority component. Sample preparation may include any known method of concentrating components, such as microbes or nucleic acids, from a larger sample volume, such as by filtration of airborne or waterborne particles from a larger volume sample or by isolation of microbes from a sample by using standard microbiology methods. Sample preparation may include physical disruption and/or chemical lysis of cellular components to release intracellular components into a substantially aqueous or organic phase and removal of debris, such as by using filtration, centrifugation or adsorption. Sample preparation may include use of a nucleic acid oligonucleotide that selectively or non-specifically capture a target nucleic acid and separate it from other sample components (e.g., as described in U.S. Pat. No. 6,110,678 and International Patent Application Pub. No. WO 2008/016988, each incorporated by reference herein).

“Separating” or “purifying” means that one or more components of a sample are removed or separated from other sample components. Sample components include target nucleic acids usually in a generally aqueous solution phase, which may also include cellular fragments, proteins, carbohydrates, lipids, and other nucleic acids. “Separating” or “purifying” does not connote any degree of purification. Typically, separating or purifying removes at least 70%, or at least 80%, or at least 95% of the target nucleic acid from other sample components.

The term “specificity,” in the context of an amplification and/or detection system, is used herein to refer to the characteristic of the system which describes its ability to distinguish between target and non-target sequences dependent on sequence and assay conditions. In terms of nucleic acid amplification, specificity generally refers to the ratio of the number of specific amplicons produced to the number of side-products (e.g., the signal-to-noise ratio). In terms of detection, specificity generally refers to the ratio of signal produced from target nucleic acids to signal produced from non-target nucleic acids.

The term “sensitivity” is used herein to refer to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

As used herein, the term “relative light unit” (“RLU”) is an arbitrary unit of measurement indicating the relative number of photons emitted by the sample at a given wavelength or band of wavelengths. RLU varies with the characteristics of the detection means used for the measurement.

The present disclosure is generally directed to methods and combination of oligomers for detecting the presence or absence of SARS-CoV-2 in a sample, such as biological samples. In some embodiments, the present disclosure provides methods and combination of oligomers for diagnosing COVID-19 in a subject. In other, non-mutually exclusive embodiments, the present disclosure provides methods for the detection of SARS-CoV-2 in a sample, where the method includes performing amplification-based detection of a target nucleic acid from SARS-CoV-2. The present disclosure further provides compositions, reaction mixtures and kits comprising a combination of oligomers for detecting SARS-CoV-2 in a sample. The combination of oligomers generally includes at least two amplification oligomers for detecting SARS-CoV-2 in a sample, and may further include one or more additional oligomers as described herein for performing amplification-based detection of SARS-CoV-2, such as, for example, a capture probe and/or a detection probe.

The methods for diagnosing COVID-19 generally include detecting the presence or absence of SARS-CoV-2 in a sample from a subject. The sample may be suspected of being infected with or containing SARS-CoV-2. The subject may be suspected of being infected with SARS-CoV-2 or having COVID-19. In particular, an assay is performed for the specific detection in the sample of SARS-CoV-2 nucleic acid. Based on the results from the detection assay, a status of either positive or negative is assigned for the SARS-CoV-2. The presence or absence of COVID-19 in the subject can be determined based on the SARS-CoV-2 status.

While SARS-CoV-2 nucleic acid may be detected using any suitable method, it is preferred that this virus is detected using a nucleic-acid-based detection assay. Nucleic-acid-based detection assays generally utilize oligonucleotides that specifically hybridize to a target nucleic acid of SARS-CoV-2 with minimal cross-reactivity to other nucleic acids suspected of being in a sample. Accordingly, oligonucleotides for nucleic-acid-based detection of SARS-CoV-2 will have minimal cross-reactivity to other nucleic acids including, for example, SARS coronavirus and other related Sarbecovirus.

A positive signal from a nucleic-acid-based detection assay in accordance with the present disclosure is indicative of the presence of SARS-CoV-2 in a sample.

In some embodiments, a method comprising the use of a nucleic-acid-based detection assay—such as an amplification-based assay—is used to detect SARS-CoV-2. Such method generally includes amplifying a target sequence within a target nucleic acid utilizing an in vitro nucleic acid amplification reaction and detecting the amplified product by, for example, specifically hybridizing the amplified product with a nucleic acid detection probe that provides a signal to indicate the presence of a target in the sample. The amplification step includes contacting the sample with two or more amplification oligomers specific for a target sequence in a target nucleic acid to produce an amplified product if the target nucleic acid is present in the sample. Amplification synthesizes additional copies of the target sequence or its complement by using at least one nucleic acid polymerase to extend the sequence from an amplification oligomer (a primer) using a template strand. One embodiment for detecting the amplified product uses a hybridizing step that includes contacting the amplified product with at least one probe specific for a sequence amplified by the selected amplification oligomers, e.g., a sequence contained in the target sequence flanked by a pair of selected amplification oligomers. Suitable amplification methods include, for example, replicase-mediated amplification, polymerase chain reaction (PCR), ligase chain reaction (LCR), strand-displacement amplification (SDA), and transcription-mediated or transcription-associated amplification (TMA). Such amplification methods are well-known in the art (see, e.g., discussion of amplification methods above) and are readily used in accordance with the methods of the present disclosure.

For example, some amplification methods that use TMA amplification include the following steps. Briefly, the target nucleic acid that contains the sequence to be amplified is provided as single stranded nucleic acid (e.g., ssRNA or ssDNA). Those skilled in the art will appreciate that conventional melting of double stranded nucleic acid (e.g., dsDNA) may be used to provide single-stranded target nucleic acids. A promoter primer binds specifically to the target nucleic acid at its target sequence and a reverse transcriptase (RT) extends the 3′ end of the promoter primer using the target strand as a template to create a cDNA copy of the target sequence strand, resulting in an RNA:DNA duplex. An RNase digests the RNA strand of the RNA:DNA duplex and a second primer binds specifically to its target sequence, which is located on the cDNA strand downstream from the promoter primer end. RT synthesizes a new DNA strand by extending the 3′ end of the second primer using the first cDNA template to create a dsDNA that contains a functional promoter sequence. An RNA polymerase specific for the promoter sequence then initiates transcription to produce RNA transcripts that are about 100 to 1000 amplified copies (“amplicons”) of the initial target strand in the reaction. Amplification continues when the second primer binds specifically to its target sequence in each of the amplicons and RT creates a DNA copy from the amplicon RNA template to produce an RNA:DNA duplex. RNase in the reaction mixture digests the amplicon RNA from the RNA:DNA duplex and the promoter primer binds specifically to its complementary sequence in the newly synthesized DNA. RT extends the 3′ end of the promoter primer to create a dsDNA that contains a functional promoter to which the RNA polymerase binds to transcribe additional amplicons that are complementary to the target strand. The autocatalytic cycles of making more amplicon copies repeat during the course of the reaction resulting in about a billion-fold amplification of the target nucleic acid present in the sample. The amplified products may be detected in real-time during amplification, or at the end of the amplification reaction by using a probe that binds specifically to a target sequence contained in the amplified products. Detection of a signal resulting from the bound probes indicates the presence of the target nucleic acid in the sample.

In some embodiments, the method utilizes a “reverse” TMA reaction. In such variations, the initial or “forward” amplification oligomer is a priming oligonucleotide that hybridizes to the target nucleic acid in the vicinity of the 3′-end of the target region. A reverse transcriptase (RT) synthesizes a cDNA strand by extending the 3′-end of the primer using the target nucleic acid as a template. The second or “reverse” amplification oligomer is a promoter primer or promoter provider having a target-hybridizing sequence configured to hybridize to a target-sequence contained within the synthesized cDNA strand. Where the second amplification oligomer is a promoter primer, RT extends the 3′ end of the promoter primer using the cDNA strand as a template to create a second, cDNA copy of the target sequence strand, thereby creating a dsDNA that contains a functional promoter sequence. Amplification then continues essentially as described above for initiation of transcription from the promoter sequence utilizing an RNA polymerase. Alternatively, where the second amplification oligomer is a promoter provider, a terminating oligonucleotide, which hybridizes to a target sequence that is in the vicinity to the 5 ‘-end of the target region, is typically utilized to terminate extension of the priming oligomer at the 3’-end of the terminating oligonucleotide, thereby providing a defined 3′-end for the initial cDNA strand synthesized by extension from the priming oligomer. The target-hybridizing sequence of the promoter provider then hybridizes to the defined 3′-end of the initial cDNA strand, and the 3′-end of the cDNA strand is extended to add sequence complementary to the promoter sequence of the promoter provider, resulting in the formation of a double-stranded promoter sequence. The initial cDNA strand is then used a template to transcribe multiple RNA transcripts complementary to the initial cDNA strand, not including the promoter portion, using an RNA polymerase that recognizes the double-stranded promoter and initiates transcription therefrom. Each of these RNA transcripts is then available to serve as a template for further amplification from the first priming amplification oligomer.

Detection of the amplified products may be accomplished by a variety of methods to detect a signal specifically associated with the amplified target sequence. The nucleic acids may be associated with a surface that results in a physical change, such as a detectable electrical change. Amplified nucleic acids may be detected by concentrating them in or on a matrix and detecting the nucleic acids or dyes associated with them (e.g. an intercalating agent such as ethidium bromide or cyber green), or detecting an increase in dye associated with nucleic acid in solution phase. Other methods of detection may use nucleic acid detection probes oligomers that are configured to specifically hybridize to a sequence in the amplified product and detecting the presence of the probe:product complex, or by using a complex of probes that may amplify the detectable signal associated with the amplified products (e.g., U.S. Pat. Nos. 5,424,413; 5,451,503; and 5,849,481). Directly or indirectly labeled probes that specifically associate with the amplified product provide a detectable signal that indicates the presence of the target nucleic acid in the sample.

Detection probes oligomers (where labelled) that hybridize to the complementary amplified sequences may be DNA or RNA oligomers, or oligomers that contain a combination of DNA and RNA nucleotides, or oligomers synthesized with a modified backbone, e.g., an oligomer that includes one or more 2′-methoxy substituted ribonucleotides. Probes used for detection of the amplified sequences may be unlabeled and detected indirectly (e.g., by binding of another binding partner to a moiety on the probe) or may be labeled with a variety of detectable labels. In some embodiments of the method for detecting SARS-CoV-2, such as in certain embodiments using transcription-mediated amplification (TMA), the detection probe is a linear chemiluminescently labeled probe such as, e.g., a linear acridinium ester (AE) labeled probe. The detection step may also provide additional information on the amplified sequence, such as, e.g., all or a portion of its nucleic acid base sequence. Detection may be performed after the amplification reaction is completed, or may be performed simultaneously with amplifying the target region, e.g., in real time. In one embodiment, the detection step allows homogeneous detection, e.g., detection of the hybridized probe without removal of unhybridized probe from the mixture (see, e.g., U.S. Pat. Nos. 5,639,604 and 5,283,174).

In embodiments that detection of the amplified product occurs near or at the end of the amplification step, a linear detection probe may be used to provide a signal to indicate hybridization of the probe to the amplified product. One example of such detection uses a luminescentally labeled probe that hybridizes to target nucleic acid. Luminescent label is then hydrolyzed from non-hybridized probe. Detection is performed by chemiluminescence using a luminometer. (see, e.g., International Patent Application Pub. No. WO 89/002476). In other embodiments that use real-time detection, the detection probe may be a hairpin probe such as, for example, a molecular beacon, molecular torch, or hybridization switch probe that is labeled with a reporter moiety that is detected when the probe binds to amplified product. Such probes may comprise target-hybridizing sequences and non-target-hybridizing sequences. Various forms of such probes have been described previously (see, e.g., U.S. Pat. Nos. 5,118,801; 5,312,728; 5,925,517; 6,150,097; 6,849,412; 6,835,542; 6,534,274; and 6,361,945; and US Patent Application Pub. Nos. 20060068417A1 and 20060194240A1).

In certain embodiments utilizing a nucleic-acid-based detection assay, the method further includes purifying the SARS-CoV-2 target nucleic acid from other components in the sample. Such purification may include methods of separating and/or concentrating organisms contained in a sample from other sample components. In particular embodiments, purifying the target nucleic acid includes capturing the target nucleic acid to specifically or non-specifically separate the target nucleic acid from other sample components. Non-specific target capture methods may involve selective precipitation of nucleic acids from a substantially aqueous mixture, adherence of nucleic acids to a support that is washed to remove other sample components, or other means of physically separating nucleic acids from a mixture that contains SARS-CoV-2 nucleic acid and other sample components.

In some embodiments, a target nucleic acid of SARS-CoV-2 is separated from other sample components by hybridizing the target nucleic acid to a capture probe oligomer. The capture probe oligomer comprises a target-hybridizing sequence configured to specifically or non-specifically hybridize to a target nucleic acid so as to form a [target nucleic acid]:[capture probe] complex that is separated from other sample components. Capture probes comprising target-hybridizing sequences suitable for non-specific capture of target nucleic acids are described in, e.g., WO 2008/016988.

In some specific variations comprising target-hybridizing sequence(s) configured to specifically hybridize to a SARS-CoV-2 target nucleic acid, a SARS-CoV-2-specific capture probe comprises a target-hybridizing sequence. In a preferred variation, the capture probe binds the [target nucleic acid]: [capture probe] complex to an immobilized probe to form a [target nucleic acid]: [capture probe]: [immobilized probe] complex that is separated from the sample and, optionally, washed to remove non-target sample components (see, e.g., U.S. Pat. Nos. 6,110,678; 6,280,952; and 6,534,273). In such variations, the capture probe oligomer further comprises a sequence or moiety that binds the capture probe, with its bound target sequence, to an immobilized probe attached to a solid support, thereby permitting the hybridized target nucleic acid to be separated from other sample components.

In some embodiments, the capture probe oligomer includes a tail portion (e.g., a 3′ tail) that is not complementary to target nucleic acid but that specifically hybridizes to a sequence on the immobilized probe, thereby serving as the moiety allowing the target nucleic acid to be separated from other sample components, such as previously described in, e.g., U.S. Pat. No. 6,110,678. Any sequence may be used in a tail region, which is generally about 5 to 50 nucleotides long, and preferred embodiments include a substantially homopolymeric tail of about 10 to 40 nucleotides (e.g., A10 to A40), more preferably about 14 to 33 nt (e.g., A14 to A30 or T3A14 to T3A30), that bind to a complementary immobilized sequence (e.g., poly-T) attached to a solid support, e.g., a matrix or particle.

Target capture typically occurs in a solution phase mixture that contains one or more capture probe oligomers that hybridize to the target nucleic acid under hybridizing conditions, usually at a temperature higher than the Tm of the [tail sequence]: [immobilized probe sequence] duplex. For embodiments comprising a capture probe tail, the [target nucleic acid]: [capture probe] complex is captured by adjusting the hybridization conditions so that the capture probe tail hybridizes to the immobilized probe, and the entire complex on the solid support is then separated from other sample components. The support with the attached [immobilized probe]: [capture probe]: [target nucleic acid] may be washed one or more times to further remove other sample components. Preferred embodiments use a particulate solid support, such as paramagnetic beads, so that particles with the attached [target nucleic acid]: [capture probe]: [immobilized probe] complex may be suspended in a washing solution and retrieved from the washing solution, preferably by using magnetic attraction. In embodiments of the method comprising the use of an amplification-based detection assay, to limit the number of handling steps, a target nucleic acid may be amplified by simply mixing the target nucleic acid in the complex on the support with amplification oligomers and proceeding with amplification steps.

In accordance with the present invention, detecting the presence or absence of SARS-CoV-2 may be performed separately (e.g., in a separate reaction vessel), or performed together with another assay as a multiplex reaction system. Accordingly, in some embodiments, a method as described herein (e.g., a method for diagnosing COVID-19) utilizes a multiplex reaction, where the reaction mix contains reagents for assaying multiple (e.g., at least two, three, four, or more) different target sequences in parallel. In these cases, a reaction mix may contain multiple different target-specific oligonucleotides for performing the detection assay. For example, in a method utilizing an amplification-based detection assay, a multiplex reaction may contain multiple sets (e.g., multiple pairs) of amplification oligomers (for example, multiple pairs of PCR primers or multiple pairs of TMA amplification oligomers (e.g., for TMA, multiple pairs of promoter primer and non-promoter primer, or multiple pairs of promoter provider and non-promoter primer)).

In one aspect, the present invention provides a method for detecting SARS-CoV-2 nucleic acid in a sample, said method comprising:
(1) contacting a sample, said sample suspected of containing SARS-CoV-2 nucleic acid, with at least two amplification oligomers for amplifying at least one target region of a SARS-CoV-2 target nucleic acid, wherein said at least two amplification oligomers comprise:
   (I) a first and a second amplification oligomers for amplifying a first target region of a SARS-CoV-2 nucleic acid, wherein
      (a) the first amplification oligomer comprises a first target-hybridizing sequence comprising or consisting of a sequence selected from the group consisting of SEQ ID NO:2, SEQ ID NO:4 and SEQ ID NO:23; and
      (b) the second amplification oligomer comprises a second target-hybridizing sequence comprising or consisting of a sequence selected from the group consisting of SEQ ID NO:5, and SEQ ID NO:6;
      and/or
   (II) a first and a second amplification oligomers for amplifying a second target region of a SARS-CoV-2 nucleic acid, wherein
      (a) the first amplification oligomer comprises a first target-hybridizing sequence comprising or consisting of a sequence selected from the group consisting of SEQ ID NO:27 and SEQ ID NO:29; and
      (b) the second amplification oligomer comprises a second target-hybridizing sequence comprising or consisting of a sequence selected from the group consisting of SEQ ID NO:30 and SEQ ID NO:31;
(2) performing an in vitro nucleic acid amplification reaction, wherein any SARS-CoV-2 target nucleic acid present in said sample is used as a template for generating an amplification product; and
(3) detecting the presence or absence of the amplification product, thereby indicating the presence or absence of SARS-CoV-2 target nucleic acid in said sample.

In some embodiments of the methods, the first amplification oligomer for amplifying the first target region comprises or consists of a sequence selected from the group consisting of SEQ ID NO:1, SEQ ID NO:2, SEQ ID NO:3, SEQ ID NO:4, SEQ ID NO: 22 and SEQ ID NO:23.

In some embodiments, the second amplification oligomer for amplifying the first target region comprises or consists of a sequence selected from the group consisting of SEQ ID NO:5, and SEQ ID NO:6.

In some embodiments, the first amplification oligomer for amplifying the second target region comprises or consists of a sequence selected from the group consisting of SEQ ID NO:26, SEQ ID NO:27, SEQ ID NO:28 and SEQ ID NO:29.

In some embodiments, the second amplification oligomer for amplifying the second target region comprises or consists of a sequence selected from the group consisting of SEQ ID NO:30, and SEQ ID NO:31.

In some embodiments, the first amplification oligomer for amplifying the first and/or the second target region is a promoter primer or promoter provider further comprising a promoter sequence located 5′ to the first target-hybridizing sequence.

In some embodiments, the promoter sequence is a T7 promoter sequence.

In some embodiments, the T7 promoter sequence comprises or consists of SEQ ID NO: 7.

In some embodiments, the first and second target-hybridizing sequences of the first and second amplification oligomers for amplifying the first target region respectively comprise or consist of the nucleotide sequences of:
(a) SEQ ID NO:2, SEQ ID NO:4 or SEQ ID NO:23 and SEQ ID NO:5; or
(b) SEQ ID NO:2 SEQ ID NO:4 or SEQ ID NO:23 and SEQ ID NO:6; or
(c) SEQ ID NO:2 and SEQ ID NO:5 or SEQ ID NO:6; or
(d) SEQ ID NO:4 and SEQ ID NO:5 or SEQ ID NO:6; or
(e) SEQ ID NO:23 and SEQ ID NO:5 or SEQ ID NO:6; or
(f) SEQ ID NO:2 and SEQ ID NO:5; or
(g) SEQ ID NO:2 and SEQ ID NO:6; or
(h) SEQ ID NO:4 and SEQ ID NO:5; or
(i) SEQ ID NO:4 and SEQ ID NO:6, or
(j) SEQ ID NO:23 and SEQ ID NO:5; or
(k) SEQ ID NO:23 and SEQ ID NO:6.

In some embodiments, the first and second target-hybridizing sequences of the first and second amplification oligomers for amplifying the second target region respectively comprise or consist of the nucleotide sequences of:
(a) SEQ ID NO:27 or SEQ ID NO:29 and SEQ ID NO:30; or
(b) SEQ ID NO:27 or SEQ ID NO:29 and SEQ ID NO:31; or
(c) SEQ ID NO:27 and SEQ ID NO:30 or SEQ ID NO:31; or
(d) SEQ ID NO:29 and SEQ ID NO:30 or SEQ ID NO:31; or
(e) SEQ ID NO:27 and SEQ ID NO:30; or
(f) SEQ ID NO:27 and SEQ ID NO:31; or
(g) SEQ ID NO:29 and SEQ ID NO:30; or
(h) SEQ ID NO:29 and SEQ ID NO:31.

In some embodiments, the at least two amplification oligomers for amplifying the first target region or the second target region comprise redundant first amplification oligomers and/or redundant second amplification oligomers.

In some embodiments, the first and second target-hybridizing sequences of the redundant first and/or second amplification oligomers for amplifying the first target region respectively comprise or consist of the nucleotide sequences of:
(a) SEQ ID NO:2 and SEQ ID NO:4 and SEQ ID NO:5; or
(b) SEQ ID NO:2 and SEQ ID NO:4 and SEQ ID NO:6; or
(c) SEQ ID NO:2 and SEQ ID NO:5 and SEQ ID NO:6; or
(d) SEQ ID NO:4 and SEQ ID NO:5 and SEQ ID NO:6, or
(e) SEQ ID NO:2 and SEQ ID NO:4 and SEQ ID NO:5 and SEQ ID NO:6, or
(f) SEQ ID NO:2 and SEQ ID NO:23 and SEQ ID NO:5; or
(g) SEQ ID NO:2 and SEQ ID NO:23 and SEQ ID NO:6; or;
(h) SEQ ID NO:23 and SEQ ID NO:5 and SEQ ID NO:6, or
(i) SEQ ID NO:2 and SEQ ID NO:23 and SEQ ID NO:5 and SEQ ID NO:6, or
(j) SEQ ID NO:4 and SEQ ID NO:23 and SEQ ID NO:5; or
(k) SEQ ID NO:4 and SEQ ID NO:23 and SEQ ID NO:6; or;
(l) SEQ ID NO:4 and SEQ ID NO:23 and SEQ ID NO:5 and SEQ ID NO:6.

In some embodiments, the first and second target-hybridizing sequences of the redundant first and/or second amplification oligomers for amplifying the second target region respectively comprise or consist of the nucleotide sequences of:
(a) SEQ ID NO:27 and SEQ ID NO:29 and SEQ ID NO:30; or
(b) SEQ ID NO:27 and SEQ ID NO:29 and SEQ ID NO:31; or
(c) SEQ ID NO:27 and SEQ ID NO:30 and SEQ ID NO:31; or
(d) SEQ ID NO:29 and SEQ ID NO:30 and SEQ ID NO:31; or
(e) SEQ ID NO:27 and SEQ ID NO:29 and SEQ ID NO:30 and SEQ ID NO:31.

In some embodiments, the at least two amplification oligomers for amplifying the first and the second target regions comprise redundant first amplification oligomers and/or redundant second amplification oligomers.

In some embodiments, the redundant first and/or second amplification oligomers for amplifying the first and the second target regions respectively comprise or consist of the nucleotide sequences of:
(a) SEQ ID NO:2, SEQ ID NO:4, SEQ ID NO:5, SEQ ID NO:6. SEQ ID NO:27, SEQ ID NO:29, SEQ ID NO:30 and SEQ ID NO:31, or
(b) SEQ ID NO:2, SEQ ID NO:23, SEQ ID NO:5. SEQ ID NO:6. SEQ ID N:27, SEQ ID NO:29, SEQ ID NO:30 and SEQ ID NO:31, or
(c) SEQ ID N:4, SEQ ID NO:23, SEQ ID NO:5, SEQ ID NO:6, SEQ ID NO:27, SEQ ID NO:29, SEQ ID NO:30 and SEQ ID NO:31

In some embodiments, the method of the present invention further comprises purifying the target nucleic acid from other components in the sample before step (1).

In some embodiments, the purifying step comprises contacting the sample with at least one capture probe oligomer comprising a target-hybridizing sequence covalently attached to a sequence or moiety that binds to an immobilized probe, wherein said target-hybridizing sequence comprises or consists of a sequence selected from the group consisting of SEQ ID NO:9, and SEQ ID NO:11 or from the group consisting of SEQ ID NO:33, and SEQ ID NO:35.

In some embodiments, in the method of the present invention the at least one capture probe oligomer sequence comprises or consists of a sequence selected from the group consisting of SEQ ID NO:8, SEQ ID NO:9, SEQ ID NO:10, and SEQ ID NO:11 or from the group consisting of SEQ ID NO:32, SEQ ID NO:33, SEQ ID NO:34 and SEQ ID NO:35.

In some embodiments, in the method of the present invention the at least one capture probe oligomer is at least two capture probe oligomers respectively comprising or consisting of at least one sequence selected from the group consisting of SEQ ID NO:8, SEQ ID NO:9, SEQ ID NO:10, and SEQ ID NO:11 and at least one from the group consisting of SEQ ID NO:32, SEQ ID NO:33, SEQ ID NO:34, and SEQ ID NO:35.

In some embodiments, the at least two capture probe oligomers comprise or consist of SEQ ID NO:8, SEQ ID NO:10, SEQ ID NO:32 and SEQ ID NO:34 or SEQ ID NO: 9, SEQ ID NO: 11, SEQ ID NO:33; and SEQ ID NO: 35.

In some embodiments, the detecting step (3) comprises contacting said in vitro nucleic acid amplification reaction with at least one detection probe oligomer hybridizes to the amplification product under conditions whereby the presence or absence of the amplification product is determined, thereby indicating the presence or absence of SARS-CoV-2 in said sample.

In some embodiments, the at least one detection probe oligomer comprises a target-hybridizing sequence that is from about 14 to about 40 nucleotides in length and hybridizes to a target sequence comprising or consisting of SEQ ID NO:13, the DNA equivalent of SEQ ID NO:13, the complement of SEQ ID NO:13, the DNA equivalent of the complement of SEQ ID NO:13, or the DNA/RNA chimeric of SEQ ID NO:13, or SEQ ID NO: 25, the RNA equivalent of SEQ ID NO:25, the complement of SEQ ID NO:25, the RNA equivalent of the complement of SEQ ID NO:25, or the DNA/RNA chimeric thereof.

In some embodiments, the detection probe target-hybridizing sequence is contained in the sequence of SEQ ID NO:25 and includes at least the sequence of SEQ ID NO:13.

In some embodiments, the detection probe oligomer comprises or consists of SEQ ID NO:13.

In some embodiments, the at least one detection probe oligomer comprises a target-hybridizing sequence that is from about 14 to about 40 nucleotides in length and hybridizes to a target sequence comprising or consisting of SEQ ID NO:36, the DNA equivalent of SEQ ID NO: 36, the complement of SEQ ID NO: 36, the DNA equivalent of the complement of SEQ ID NO: 36, or the DNA/RNA chimeric of SEQ ID NO: 36, or SEQ ID NO:43, the RNA equivalent of SEQ ID NO:43, the complement of SEQ ID NO:43, the RNA equivalent of the complement of SEQ ID NO:43, or the DNA/RNA chimeric thereof.

In some embodiments, the detection probe target-hybridizing sequence is contained in the sequence of SEQ ID NO:43 and includes at least the sequence of SEQ ID NO:36.

In some embodiments, the detection probe oligomer comprises or consists of SEQ ID NO:36.

In some embodiments, the least one detection probe oligomer is at least two different detection probe oligomers comprising respectively SEQ ID NO:13 and SEQ ID NO:36.

In some embodiments, the first and second amplification oligomer for amplifying the first target region and the detection probe oligomer respectively comprise or consist of the nucleotide sequences of:
(a) SEQ ID NO:2 and SEQ ID NO:5 and SEQ ID NO:13; or
(b) SEQ ID NO:2 and SEQ ID NO:6; and SEQ ID NO:13; or
(c) SEQ ID NO:4 and SEQ ID NO:5; and SEQ ID NO:13; or
(d) SEQ ID NO:4 and SEQ ID NO:6. and SEQ ID NO:13, or
(e) SEQ ID NO:23 and SEQ ID NO:5; and SEQ ID NO:13; or
(f) SEQ ID NO:23 and SEQ ID NO:6. and SEQ ID NO:13.

In some embodiments, the first and second amplification oligomer for amplifying the second target region and the detection probe oligomer respectively comprise or consist of the nucleotide sequences of:
(a) SEQ ID NO:27 and SEQ ID NO:30 and SEQ ID NO:36; or
(b) SEQ ID NO:27 and SEQ ID NO:31; and SEQ ID NO:36; or
(c) SEQ ID NO:29 and SEQ ID NO:30; and SEQ ID NO:36; or
(d) SEQ ID NO:29 and SEQ ID NO:31; and SEQ ID NO:36.

In some embodiments, the at least two amplification oligomers for amplifying the first and/or the second target region comprise redundant first amplification oligomers and/or redundant second amplification oligomers and/or redundant detection probe oligomers.

In some embodiments, the redundant first and/or second amplification oligomers for amplifying the first and the second target regions and/or the redundant detection probes respectively comprise or consist of the nucleotide sequences of:
(a) SEQ ID NO:2, SEQ ID NO:4, SEQ ID NO:5, SEQ ID NO:6 and SEQ NO:13, or
(b) SEQ ID NO:2, SEQ ID NO:23, SEQ ID NO:5, SEQ ID NO:6 and SEQ NO:13, or
(c) SEQ ID NO:4, SEQ ID NO:23, SEQ ID NO:5, SEQ ID NO:6 and SEQ NO:13, or
(d) SEQ ID NO:27, SEQ ID NO:29. SEQ ID NO:30, SEQ ID NO:31, and SEQ NO:36, or
(e) SEQ ID NO:2, SEQ ID NO:4, SEQ ID NO:5, SEQ ID NO:6, SEQ ID NO:27, SEQ ID NO:29, SEQ ID NO:30, SEQ ID NO:31, SEQ NO:13 and SEQ NO:36, or
(f) SEQ ID NO:2, SEQ ID NO:23, SEQ ID NO:5, SEQ ID NO:6, SEQ ID NO:27, SEQ ID NO:29, SEQ ID NO:30, SEQ ID NO:31, SEQ NO:13 and SEQ NO:36 or
(g) SEQ ID NO:4, SEQ ID NO:23, SEQ ID NO:5, SEQ ID NO:6, SEQ ID NO:27, SEQ ID NO:29, SEQ ID NO:30, SEQ ID NO:31, SEQ NO:13 and SEQ NO:36

In some embodiments, the detection probe oligomer further comprises a 2′ methoxy modification on at least one of a nucleotide residue member of the nucleotide sequence.

In some embodiments, the detection probe oligomer comprises a label selecting from the group consisting of a chemiluminescent label, a fluorescent label, quencher and a combination of one or more thereof.

In some embodiments, the label is a chemiluminescent acridinium ester (AE) compound linked between two nucleobases of the detection probe oligomer.

In some embodiments, the detecting step (3) further comprises contacting said in vitro nucleic acid amplification reaction with a probe protection oligomer substantially complementary to a detection probe oligomer to stabilize the labelled probe during storage under conditions whereby the presence or absence of the amplification product is determined, thereby indicating the presence or absence of SARS-CoV-2 in said sample.

In some embodiments, the amplification reaction at step (2) is an isothermal amplification reaction.

In some embodiments, the isothermal amplification reaction is a transcription-mediated amplification (TMA) reaction.

In some embodiments, the detection step (3) is a hybridization protection assay (HPA).

In some embodiments, the sample is a clinical sample, preferably, the sample is a blood sample, more preferably the sample is a plasma sample or a serum sample.

In some embodiments, the amplification product has a length of from about 27 to about 79 contiguous nucleotides and contains SEQ ID NO:21, or SEQ ID NO:40 or the complements thereof.

In another aspect, the present invention provides a method for detecting SARS-CoV-2 nucleic acid in a sample, said method comprising:
(1) contacting a sample, said sample suspected of containing SARS-CoV-2 nucleic acid, with at least two amplification oligomers for amplifying at least one target region of a SARS-CoV-2 target nucleic acid, wherein said at least two amplification oligomers comprise:
   (I) a first and a second amplification oligomers for amplifying a first target region of a SARS-CoV-2 nucleic acid, wherein
      (a) the first amplification oligomer comprises a first target-hybridizing sequence comprising or consisting of a sequence selected from the group consisting of SEQ ID NO:2, SEQ ID NO:4 and SEQ ID NO:23; or
      (b) the second amplification oligomer comprises a second target-hybridizing comprising or consisting of a sequence selected from the group consisting of SEQ ID NO:5 and SEQ ID NO:6;
      and/or
   (II) a first and a second amplification oligomers for amplifying a second target region of a SARS-CoV-2 nucleic acid, wherein
      (a) the first amplification oligomer comprises a first target-hybridizing sequence comprising or consisting of a sequence selected from the group consisting of SEQ ID NO:27, and SEQ ID NO:29; or
      (b) the second amplification oligomer comprises a second target-hybridizing comprising or consisting of a sequence selected from the group consisting of SEQ ID NO:30 and SEQ ID NO:31;
(2) performing an in vitro nucleic acid amplification reaction, wherein any SARS-CoV-2 target nucleic acid present in said sample is used as a template for generating an amplification product; and
(3) detecting the presence or absence of the amplification product, thereby indicating the presence or absence of SARS-CoV-2 target nucleic acid in said sample.

In some embodiments, said amplification product has a length of from 65 to 119 contiguous nucleotides and contains SEQ ID NO:21, or SEQ ID NO:40 or the complements thereof.

In some embodiments, the first amplification oligomer for amplifying the first target region comprises or consists of a sequence selected from the group consisting of SEQ ID NO:1, SEQ ID NO:2, SEQ ID NO:3, SEQ ID NO:4, SEQ ID NO:22 and SEQ ID NO:23.

In some embodiments, the second amplification oligomer for amplifying the first target region comprises or consists of a sequence selected from the group consisting of SEQ ID NO:5, and SEQ ID NO:6.

In some embodiments, the first amplification oligomer for amplifying the second target region comprises or consists of a sequence selected from the group consisting of SEQ ID NO:26, SEQ ID NO:27, SEQ ID NO:28 and SEQ ID NO:29.

In some embodiments, the second amplification oligomer for amplifying the second target region comprises or consists of a sequence selected from the group consisting of SEQ ID NO:30, and SEQ ID NO:31.

In some embodiments, the first amplification oligomer for amplifying the first and/or the second target region is a promoter primer or promoter provider further comprising a promoter sequence located 5′ to the first target-hybridizing sequence.

In some embodiments, the promoter sequence is a T7 promoter sequence.

In some embodiments, the T7 promoter sequence comprises or consists of SEQ ID NO:7.

In some embodiments, the at least two amplification oligomers for amplifying the first target region and/or the second target region comprise redundant first amplification oligomers and/or redundant second amplification oligomers.

In some embodiments, the redundant oligomers are the following groups:
(a) SEQ ID NO:2 and SEQ ID NO:4; or
(b) SEQ ID NO:2 and SEQ ID NO:23; or
(c) SEQ ID NO:4 and SEQ ID NO:23; or
(d) SEQ ID NO:5 and SEQ ID NO:6, or
(e) SEQ ID NO:27 and SEQ ID NO:29, or
(f) SEQ ID: NO:30 and SEQ ID NO:31

In some embodiments, the method of the present invention further comprises purifying the target nucleic acid from other components in the sample before step (1).

In some embodiments, the purifying step comprises contacting the sample with at least one capture probe oligomer comprising a target-hybridizing sequence covalently attached to a sequence or moiety that binds to an immobilized probe, wherein said target-hybridizing sequence comprises or consists of a sequence selected from the group consisting of SEQ ID NO:9, and SEQ ID NO:11 or from the group consisting of SEQ ID NO:33, and SEQ ID NO:35.

In some embodiments, the capture probe oligomer sequence comprises or consists of a sequence selected from the group consisting of SEQ ID NO:8, SEQ ID NO:9, SEQ ID NO:10, and SEQ ID NO:11 or from the group consisting of SEQ ID NO:32, SEQ ID NO:33, SEQ ID NO:34 and SEQ ID NO:35.

In some embodiments, the at least one capture probe oligomer is at least two capture probe oligomers respectively comprising or consisting of at least one sequence selected from the group consisting of SEQ ID NO:8, SEQ ID NO:9, SEQ ID NO:10, and SEQ ID NO: 11, and at least one from the group consisting of SEQ ID NO:32, SEQ ID NO:33, SEQ ID NO:34, and SEQ ID NO: 35.

In some embodiments, the at least two capture probe oligomers comprise or consist of SEQ ID NO:8, SEQ ID NO:10, SEQ ID NO:32 and SEQ ID NO:34 or SEQ ID NO: 9, SEQ ID NO: 11, SEQ ID NO:33; and SEQ ID NO: 35.

In some embodiments, the detecting step (3) comprises contacting said in vitro nucleic acid amplification reaction with at least one detection probe oligomer configured to specifically hybridize to the amplification product under conditions whereby the presence or absence of the amplification product is determined, thereby indicating the presence or absence of SARS-CoV-2 in said sample.

In some embodiments, the at least one detection probe oligomer comprises a target-hybridizing sequence that is from about 14 to about 40 nucleotides in length hybridizes to a target sequence comprising or consisting of SEQ ID NO:13, the DNA equivalent of SEQ ID NO:13, the complement of SEQ ID NO:13, the DNA equivalent of the complement of SEQ ID NO:13, or the DNA/RNA chimeric of SEQ ID NO:13, or SEQ ID NO:25, the RNA equivalent of SEQ ID NO:25, the complement of SEQ ID NO:25, the RNA equivalent of the complement of SEQ ID NO:25, or the DNA/RNA chimeric thereof.

In some embodiments, the detection probe target-hybridizing sequence is contained in the sequence of SEQ ID NO:17 and includes at least the sequence of SEQ ID NO:13.

In some embodiments, the detection probe oligomer comprises or consists of SEQ ID NO:13.

In some embodiments, the at least one detection probe oligomer comprises a target-hybridizing sequence that is from about 14 to about 40 nucleotides in length and is configured to specifically hybridize to a target sequence comprising or consisting of SEQ ID NO:36, the DNA equivalent of SEQ ID NO: 36, the complement of SEQ ID NO: 36, the DNA equivalent of the complement of SEQ ID NO: 36, or the DNA/RNA chimeric of SEQ ID NO: 36, or SEQ ID NO:43, the RNA equivalent of SEQ ID NO:43, the complement of SEQ ID NO:43, the RNA equivalent of the complement of SEQ ID NO:43, or the DNA/RNA chimeric thereof.

In some embodiments, the detection probe target-hybridizing sequence is contained in the sequence of SEQ ID NO:41 and includes at least the sequence of SEQ ID NO:36.

In some embodiments, the detection probe oligomer comprises or consists of SEQ ID NO:36.

In some embodiments, the at least one detection probe oligomer is at least two different detection probe oligomers comprising respectively SEQ ID NO:13 and SEQ ID NO:36.

In some embodiments, the first and second amplification oligomer for amplifying the target region and the detection probe oligomer respectively comprise or consist of the nucleotide sequences of:
(a) SEQ ID NO:2 and SEQ ID NO:5 and SEQ ID NO:13; or
(b) SEQ ID NO:2 and SEQ ID NO:6; and SEQ ID NO:13; or
(c) SEQ ID NO:4 and SEQ ID NO:5; and SEQ ID NO:13; or
(d) SEQ ID NO:4 and SEQ ID NO:6. and SEQ ID NO:13, or
(e) SEQ ID NO:23 and SEQ ID NO:5; and SEQ ID NO:13; or
(f) SEQ ID NO:23 and SEQ ID NO:6. and SEQ ID NO:13.

In some embodiments, the first and second amplification oligomer for amplifying the second target region and the detection probe oligomer respectively comprise or consist of the nucleotide sequences of:
(a) SEQ ID NO:27 and SEQ ID NO:30 and SEQ ID NO:36; or
(b) SEQ ID NO:27 and SEQ ID NO:31; and SEQ ID NO:36; or
(c) SEQ ID NO:29 and SEQ ID NO:30; and SEQ ID NO:36; or
(d) SEQ ID NO:29 and SEQ ID NO:31, and SEQ ID NO:36.

In some embodiments, the at least two amplification oligomers for amplifying the first and the second target regions comprise redundant first amplification oligomers and/or redundant second amplification oligomers and/or redundant detection probe oligomers.

In some embodiments, the redundant first and/or second amplification oligomers for amplifying the first and the second target regions and/or the redundant detection probes respectively comprise or consist of the nucleotide sequences of:
(a) SEQ ID NO:2, SEQ ID NO:4, SEQ ID NO:5, SEQ ID NO:6 and SEQ NO:13, or
(b) SEQ ID NO:2, SEQ ID NO:23, SEQ ID NO:5, SEQ ID NO:6 and SEQ NO:13, or
(c) SEQ ID NO:4, SEQ ID NO:23, SEQ ID NO:5, SEQ ID NO:6 and SEQ NO:13, or
(d) SEQ ID NO:27, SEQ ID NO:29. SEQ ID NO:30, SEQ ID NO:31, and SEQ NO:36, or
(e) SEQ ID NO:2, SEQ ID NO:4, SEQ ID NO:5, SEQ ID NO:6, SEQ ID NO:27, SEQ ID NO:29, SEQ ID NO:30, SEQ ID NO:31, SEQ NO:13 and SEQ NO:36, or
(f) SEQ ID NO:2, SEQ ID NO:23, SEQ ID NO:5, SEQ ID NO:6, SEQ ID NO:27, SEQ ID NO:29, SEQ ID NO:30, SEQ ID NO:31, SEQ NO:13 and SEQ NO:36 or
(g) SEQ ID NO:4, SEQ ID NO:23, SEQ ID NO:5, SEQ ID NO:6, SEQ ID NO:27, SEQ ID NO:29, SEQ ID NO:30, SEQ ID NO:31, SEQ NO:13 and SEQ NO:36.

In some embodiments, the detection probe oligomer further comprises a 2′ methoxy modification on at least one of a nucleotide residue member of the nucleotide sequence.

In some embodiments, the detection probe oligomer comprises a label selecting from the group consisting of a chemiluminescent label, a fluorescent label, quencher and a combination of one or more thereof.

In some embodiments, the label is a chemiluminescent acridinium ester (AE) compound linked between two nucleobases of the detection probe oligomer.

In some embodiments, the detecting step (3) further comprises contacting said in vitro nucleic acid amplification reaction with a probe protection oligomer substantially complementary to a detection probe oligomer to stabilize the labelled probe during storage under conditions whereby the presence or absence of the amplification product is determined, thereby indicating the presence or absence of SARS-CoV-2 in said sample.

In some embodiments, the amplification reaction at step (2) is an isothermal amplification reaction.

In some embodiments, the isothermal amplification reaction is a transcription-mediated amplification (TMA) reaction.

In some embodiments, the detection step (3) is a hybridization protection assay (HPA).

In some embodiments, the sample is a clinical sample, preferably the sample is a blood sample, more preferably the sample is a plasma sample or a serum sample.

In some embodiments, the amplification product has a length of from about 27 to about 79 contiguous nucleotides and contains SEQ ID NO:21 or SEQ ID NO:40 or the complements thereof.

In another aspect, the present invention provides a combination of at least two oligomers for determining the presence or absence of SARS-CoV-2 in a sample, said oligomer combination comprising first and second amplification oligomers for amplifying at least one target region of SARS-CoV-2 target nucleic acid, wherein said at least two amplification oligomers comprise:
(I) a first and a second amplification oligomers for amplifying a first target region of a SARS-CoV-2 nucleic acid, wherein
   (a) the first amplification oligomer comprises a first target-hybridizing sequence comprising or consisting of a sequence selected from the group consisting of SEQ ID NO:2, SEQ ID NO:4 and SEQ ID NO:23 and/or;
   (b) the second amplification oligomer comprising a second target-hybridizing sequence comprising or consisting of a sequence selected from the group consisting of SEQ ID NO:5 and SEQ ID NO:6, and/or
(II) a first and a second amplification oligomers for amplifying a second target region of a SARS-CoV-2 nucleic acid, wherein
   (a) the first amplification oligomer comprises a first target-hybridizing sequence comprising or consisting of a sequence selected from the group consisting of SEQ ID NO:27 and SEQ ID NO:29; and/or
   (b) the second amplification oligomer comprises a second target-hybridizing sequence comprising or consisting of a sequence selected from the group consisting of SEQ ID NO:30, SEQ ID NO:31.

In some embodiments, the first amplification oligomer for amplifying the first target region comprises or consists of a sequence selected from the group consisting of SEQ ID NO:1, SEQ ID NO:2, SEQ ID NO:3, SEQ ID NO:4, SEQ ID NO: 22 and SEQ ID NO:23.

In some embodiments, the second amplification oligomer for amplifying the first target region comprises or consists of a sequence selected from the group consisting of SEQ ID NO:5, and SEQ ID NO:6.

In some embodiments, the first amplification oligomer for amplifying the second target region comprises or consists of a sequence selected from the group consisting of SEQ ID NO:26, SEQ ID NO:27, SEQ ID NO:28 and SEQ ID NO:29.

In some embodiments, the second amplification oligomer for amplifying the second target region comprises or consists of a sequence selected from the group consisting of SEQ ID NO:30, and SEQ ID NO:31.

In some embodiments, the first amplification oligomer for amplifying the first and/or the second target region is a promoter primer or promoter provider further comprising a promoter sequence located 5′ to the first target-hybridizing sequence.

In some embodiments, the promoter sequence is a T7 promoter sequence, preferably the T7 promoter sequence comprises or consists of SEQ ID NO:7.

In some embodiments, the first and second target-hybridizing sequences of the first and second amplification oligomers for amplifying the first target region respectively comprise or consist of the nucleotide sequences of:
(a) SEQ ID NO:2, SEQ ID NO:4 or SEQ ID NO:23 and SEQ ID NO:5; or
(b) SEQ ID NO:2 SEQ ID NO:4 or SEQ ID NO:23 and SEQ ID NO:6; or
(c) SEQ ID NO:2 and SEQ ID NO:5 or SEQ ID NO:6; or
(d) SEQ ID NO:4 and SEQ ID NO:5 or SEQ ID NO:6; or
(e) SEQ ID NO:23 and SEQ ID NO:5 or SEQ ID NO:6; or
(f) SEQ ID NO:2 and SEQ ID NO:5; or
(g) SEQ ID NO:2 and SEQ ID NO:6; or
(h) SEQ ID NO:4 and SEQ ID NO:5; or
(i) SEQ ID NO:4 and SEQ ID NO:6, or
(j) SEQ ID NO:23 and SEQ ID NO:5; or
(k) SEQ ID NO:23 and SEQ ID NO:6.

In some embodiments, the first and second target-hybridizing sequences of the first and second amplification oligomers for amplifying the second target region respectively comprise or consist of the nucleotide sequences of:
(a) SEQ ID NO:27 or SEQ ID NO:29 and SEQ ID NO:30; or
(b) SEQ ID NO:27 or SEQ ID NO:29 and SEQ ID NO:31; or
(c) SEQ ID NO:27 and SEQ ID NO:30 or SEQ ID NO:31; or
(d) SEQ ID NO:29 and SEQ ID NO:30 or SEQ ID NO:31; or
(e) SEQ ID NO:27 and SEQ ID NO:30; or
(f) SEQ ID NO:27 and SEQ ID NO:31; or
(g) SEQ ID NO:29 and SEQ ID NO:30; or
(h) SEQ ID NO:29 and SEQ ID NO:31.

In some embodiments, the at least two amplification oligomers for amplifying the first target region or the second target region comprise redundant first amplification oligomers and/or redundant second amplification oligomers.

In some embodiments, the first and second target-hybridizing sequences of the redundant first and/or second amplification oligomers for amplifying the first target region respectively comprise or consist of the nucleotide sequences of:
(a) SEQ ID NO:2 and SEQ ID NO:4 and SEQ ID NO:5; or
(b) SEQ ID NO:2 and SEQ ID NO:4 and SEQ ID NO:6; or
(c) SEQ ID NO:2 and SEQ ID NO:5 and SEQ ID NO:6; or
(d) SEQ ID NO:4 and SEQ ID NO:5 and SEQ ID NO:6, or
(e) SEQ ID NO:2 and SEQ ID NO:4 and SEQ ID NO:5 and SEQ ID NO:6, or
(f) SEQ ID NO:2 and SEQ ID NO:23 and SEQ ID NO:5; or
(g) SEQ ID NO:2 and SEQ ID NO:23 and SEQ ID NO:6; or;
(h) SEQ ID NO:23 and SEQ ID NO:5 and SEQ ID NO:6, or
(i) SEQ ID NO:2 and SEQ ID NO:23 and SEQ ID NO:5 and SEQ ID NO:6, or
(j) SEQ ID NO:4 and SEQ ID NO:23 and SEQ ID NO:5; or
(k) SEQ ID NO:4 and SEQ ID NO:23 and SEQ ID NO:6; or;
(l) SEQ ID NO:4 and SEQ ID NO:23 and SEQ ID NO:5 and SEQ ID NO:6.

In some embodiments, the first and second target-hybridizing sequences of the redundant first and/or second amplification oligomers for amplifying the second target region respectively comprise or consist of the nucleotide sequences of:
(a) SEQ ID NO:27 and SEQ ID NO:29 and SEQ ID NO:30; or
(b) SEQ ID NO:27 and SEQ ID NO:29 and SEQ ID NO:31; or
(c) SEQ ID NO:27 and SEQ ID NO:30 and SEQ ID NO:31; or
(d) SEQ ID NO:29 and SEQ ID NO:30 and SEQ ID NO:31; or
(e) SEQ ID NO:27 and SEQ ID NO:29 and SEQ ID NO:30 and SEQ ID NO:31.

In some embodiments, the at least two amplification oligomers for amplifying the first and the second target regions comprise redundant first amplification oligomers and/or redundant second amplification oligomers.

In some embodiments, the redundant first and/or second amplification oligomers for amplifying the first and the second target regions respectively comprise or consist of the nucleotide sequences of:
(a) SEQ ID NO:2, SEQ ID NO:4, SEQ ID NO:5, SEQ ID NO:6. SEQ ID NO:27, SEQ ID NO:29, SEQ ID NO:30 and SEQ ID NO:31, or
(b) SEQ ID NO:2, SEQ ID NO:23, SEQ ID NO:5. SEQ ID NO:6. SEQ ID N:27, SEQ ID NO:29, SEQ ID NO:30 and SEQ ID NO:31, or
(c) SEQ ID N:4, SEQ ID NO:23, SEQ ID NO:5, SEQ ID NO:6, SEQ ID NO:27, SEQ ID NO:29, SEQ ID NO:30 and SEQ ID NO:31

In some embodiments, the combination of the present invention further comprises at least one capture probe oligomer.

In some embodiments, the at least one capture probe oligomer comprises a target-hybridizing sequence covalently attached to a sequence or moiety that binds to an immobilized probe, wherein said target-hybridizing sequence comprises or consists of a sequence selected from the group consisting of SEQ ID NO:9, and SEQ ID NO:11 or from the group consisting of SEQ ID NO:33, and SEQ ID NO:35.

In some embodiments, the at least one capture probe oligomer sequence comprises or consists of a sequence selected from the group consisting of SEQ ID NO:8, SEQ ID NO:9, SEQ ID NO:10, and SEQ ID NO:11 or from the group consisting of SEQ ID NO:32, SEQ ID NO:33, SEQ ID NO:34 and SEQ ID NO:35.

In some embodiments, the at least one capture probe oligomer is at least two capture probe oligomers respectively comprising or consisting of a sequence selected from the group consisting of at least one sequence selected from the group consisting of SEQ ID NO:8, SEQ ID NO:9, SEQ ID NO:10, and SEQ ID NO: 11 and at least one from the group consisting of SEQ ID NO:32, SEQ ID NO:33, SEQ ID NO:34, and SEQ ID NO:35.

In some embodiments, the at least two capture probe oligomers comprise or consist of SEQ ID NO:8, SEQ ID NO:10, SEQ ID NO:32 and SEQ ID NO:34 or SEQ ID NO: 9, SEQ ID NO: 11, SEQ ID NO:33; and SEQ ID NO: 35.

In some embodiments, the combination of the present invention further comprises at least one detection probe oligomer.

In some embodiments, in the combination of the present invention the at least one detection probe oligomer comprises a target-hybridizing sequence that is from about 14 to about 40 nucleotides in length and hybridizes to a target sequence comprising or consisting of SEQ ID NO:13, the DNA equivalent of SEQ ID NO:13, the complement of SEQ ID NO:13, the DNA equivalent of the complement of SEQ ID NO:13, or the DNA/RNA chimeric of SEQ ID NO:13, or SEQ ID NO:25, the RNA equivalent of SEQ ID NO:25, the complement of SEQ ID NO:25, the RNA equivalent of the complement of SEQ ID NO:25, or the DNA/RNA chimeric thereof.

In some embodiments, the detection probe target-hybridizing sequence is contained in the sequence of SEQ ID NO:25 and includes at least the sequence of SEQ ID NO:13.

In some embodiments, the detection probe oligomer comprises or consists of SEQ ID NO:13.

In some embodiments, the detection probe oligomer comprises a nucleotide sequence that is from 16 to 40 contiguous nucleotides in length and specifically hybridizes to SEQ ID NO:17, the RNA equivalent of SEQ ID NO:17, the complement of SEQ ID NO:17, the RNA equivalent of the complement of SEQ ID NO:17, or the DNA/RNA chimeric thereof.

In some embodiments, the at least one detection probe oligomer comprises a target-hybridizing sequence that is from about 14 to about 40 nucleotides in length and hybridizes to a target sequence comprising or consisting of SEQ ID NO:36, the DNA equivalent of SEQ ID NO: 36, the complement of SEQ ID NO: 36, the DNA equivalent of the complement of SEQ ID NO: 36, or the DNA/RNA chimeric of SEQ ID NO: 36, or SEQ ID NO:43, the RNA equivalent of SEQ ID NO:43, the complement of SEQ ID NO:43, the RNA equivalent of the complement of SEQ ID NO:43, or the DNA/RNA chimeric thereof.

In some embodiments, the detection probe target-hybridizing sequence is contained in the sequence of SEQ ID NO:41 and includes at least the sequence of SEQ ID NO:36.

In some embodiments, the detection probe oligomer comprises or consists of SEQ ID NO:36.

In some embodiments, the least one detection probe oligomer is at least two different detection probe oligomers comprising respectively SEQ ID NO:13 and SEQ ID NO:36.

In some embodiments, the first and second amplification oligomer for amplifying the first target region and the detection probe oligomer respectively comprise or consist of the nucleotide sequences of:
(a) SEQ ID NO:2 and SEQ ID NO:5 and SEQ ID NO:13; or
(b) SEQ ID NO:2 and SEQ ID NO:6; and SEQ ID NO:13; or
(c) SEQ ID NO:4 and SEQ ID NO:5; and SEQ ID NO:13; or
(d) SEQ ID NO:4 and SEQ ID NO:6. and SEQ ID NO:13, or
(e) SEQ ID NO:23 and SEQ ID NO:5; and SEQ ID NO:13; or
(f) SEQ ID NO:23 and SEQ ID NO:6. and SEQ ID NO:13.

In some embodiments, the first and second amplification oligomer for amplifying the second target region and the detection probe oligomer respectively comprise or consist of the nucleotide sequences of:
(a) SEQ ID NO:27 and SEQ ID NO:30 and SEQ ID NO:36; or
(b) SEQ ID NO:27 and SEQ ID NO:31; and SEQ ID NO:36; or
(c) SEQ ID NO:29 and SEQ ID NO:30; and SEQ ID NO:36; or
(d) SEQ ID NO:29 and SEQ ID NO:31. and SEQ ID NO:36.

In some embodiments, the at least two amplification oligomers for amplifying the first and/or the second target region comprise redundant first amplification oligomers and/or redundant second amplification oligomers and/or redundant detection probe oligomers.

In some embodiments, the redundant first and/or second amplification oligomers for amplifying the first and the second target regions and/or the redundant detection probes respectively comprise or consist of the nucleotide sequences of:
(a) SEQ ID NO:2, SEQ ID NO:4, SEQ ID NO:5, SEQ ID NO:6 and SEQ NO:13, or
(b) SEQ ID NO:2, SEQ ID NO:23, SEQ ID NO:5, SEQ ID NO:6 and SEQ NO:13, or
(c) SEQ ID NO:4, SEQ ID NO:23, SEQ ID NO:5, SEQ ID NO:6 and SEQ NO:13, or
(d) SEQ ID NO:27, SEQ ID NO:29. SEQ ID NO:30, SEQ ID NO:31, and SEQ NO:36, or
(e) SEQ ID NO:2, SEQ ID NO:4, SEQ ID NO:5, SEQ ID NO:6, SEQ ID NO:27, SEQ
ID NO:29, SEQ ID NO:30, SEQ ID NO:31, SEQ NO:13 and SEQ NO:36, or
(f) SEQ ID NO:2, SEQ ID NO:23, SEQ ID NO:5, SEQ ID NO:6, SEQ ID NO:27, SEQ
ID NO:29, SEQ ID NO:30, SEQ ID NO:31, SEQ NO:13 and SEQ NO:36 or
(g) SEQ ID NO:4, SEQ ID NO:23, SEQ ID NO:5, SEQ ID NO:6, SEQ ID NO:27, SEQ ID NO:29, SEQ ID NO:30, SEQ ID NO:31, SEQ NO:13 and SEQ NO:36.

In some embodiments, the detection probe oligomer further comprises a 2′ methoxy modification on at least one of a nucleotide residue member of the nucleotide sequence.

In some embodiments, the detection probe oligomer comprises a label selecting from the group consisting of a chemiluminescent label, a fluorescent label, quencher and a combination of one or more thereof.

In some embodiments, the label is a chemiluminescent acridinium ester (AE) compound linked between two nucleobases of the detection probe oligomer.

In some embodiments, the combination further comprises a probe protection oligomer substantially complementary to a detection probe oligomer.

In another aspect, the present invention provides a detection probe oligomer for specifically detecting a SARS-CoV-2 target nucleic acid in a sample, said detection probe oligomer comprising a target-hybridizing sequence that is from about 14 to about 40 nucleotides in length and is configured to specifically hybridize to a target sequence comprising or consisting of SEQ ID NO:13 or SEQ ID NO: 36, the DNA equivalent of SEQ ID NO:13 or SEQ ID NO: 36, the complement of SEQ ID NO:13 or SEQ ID NO: 36, the DNA equivalent of the complement of SEQ ID NO:13 or SEQ ID NO: 36, or the DNA/RNA chimeric of SEQ ID NO:13 or SEQ ID NO: 36, or SEQ ID NO:25 or SEQ ID NO: 43, the RNA equivalent of SEQ ID NO:25 or SEQ ID NO: 43, the complement of SEQ ID NO:25 or SEQ ID NO: 43, the RNA equivalent of the complement of SEQ ID NO:25 or SEQ ID NO: 43, or the DNA/RNA chimeric thereof.

In some embodiments, the detection probe target-hybridizing sequence is contained in the sequence of SEQ ID NO:17 or SEQ ID NO: 41, and includes at least the sequence of SEQ ID NO:13 or SEQ ID NO: 36.

In some embodiments, the detection probe oligomer comprises a nucleotide sequence that is from 16 to 40 contiguous nucleotides in length and specifically hybridizes to SEQ ID NO:17 or SEQ ID NO: 41, the RNA equivalent of SEQ ID NO:17 or SEQ ID NO: 41, the complement of SEQ ID NO:17 or SEQ ID NO: 41, the RNA equivalent of the complement of SEQ ID NO:17 or SEQ ID NO: 41, or the DNA/RNA chimeric thereof.

In some embodiments, the detection probe oligomer further comprises a 2′ methoxy modification on at least one of a nucleotide residue member of the nucleotide sequence.

In some embodiments, the detection probe oligomer comprises a label selecting from the group consisting of a chemiluminescent label, a fluorescent label, quencher and a combination of one or more thereof.

In some embodiments, the label is a chemiluminescent acridinium ester (AE) compound linked between two nucleobases of the detection probe oligomer.

In another aspect, the present invention provides a capture probe oligomer for specifically isolating SARS-CoV-2 nucleic acid from a sample, said capture probe oligomer comprising a target-hybridizing sequence covalently attached to a sequence or moiety that binds to an immobilized probe, wherein said target-hybridizing sequence comprises or consists of a sequence selected from the group consisting of SEQ ID NO:9, and SEQ ID NO:11 or from the group consisting of SEQ ID NO:33, and SEQ ID NO:35.

In some embodiments, the capture probe oligomer sequence comprises or consists of a sequence selected from the group consisting of SEQ ID NO:8, SEQ ID NO:9, SEQ ID NO:10, and SEQ ID NO:11 or from the group consisting of SEQ ID NO:32, SEQ ID NO:33, SEQ ID NO:34 and SEQ ID NO:35.

In some embodiments, the capture probe oligomer sequence comprises or consists of at least one sequence selected from the group consisting of SEQ ID NO:8, SEQ ID NO:9, SEQ ID NO:10, and SEQ ID NO:11 or at least one from the group consisting of SEQ ID NO:32, SEQ ID NO:33, SEQ ID NO:34, and SEQ ID NO:35.

In another aspect, the present invention provides a composition comprising the combination of at least two of the above-mentioned oligomers.

In another aspect, the present invention provides a kit comprising the combination of at least two of the above-mentioned oligomers.

In another aspect, the present invention provides a reaction mixture comprising the combination of at least two of the above-mentioned oligomers.

In a further aspect, the present invention provides the use of the combination of at least two of the above-mentioned oligomers for specifically amplifying SARS-CoV-2 nucleic acid in a sample.

In another aspect, the present invention provides the use of the detection probe oligomer as mentioned above for specifically detecting SARS-CoV-2 nucleic acid in a sample.

In another aspect, the present invention provides the use of the capture probe oligomer as mentioned above for specifically capturing SARS-CoV-2 nucleic acid from a sample.

Finally, the present invention provides a method for diagnosing COVID-19 in a subject comprising detecting the presence of SARS-CoV-2 in a sample from said subject according to the method mentioned above.

### EXAMPLES

SARS-CoV-2 Assay

The oligomers listed in Table 1 were identified for the SARS-CoV-2 assay that was used in the examples described herein.

Assay reagents included the following: Target Capture Reagent (TCR) comprising the two capture probe oligomers listed in Table 1 (also referred as “Target Capture Oligo” or “TCO”); an Amplification Reagent comprising the T7 promoter providers and a non-T7 primers listed in Table 1; a Probe Reagent consisting of acridinium-ester (AE) labeled detection probes. Enzyme Reagent, Selection Reagent, negative and positive calibrators and internal controls reagents were also used.

“Target Capture Reagent” generally refers to a solution containing a number of components that facilitate capture of a nucleic acid from a solution. For example, the Target Capture Reagent may comprises HEPES, lithium hydroxide, lithium chloride, EDTA, at pH 6.4, and magnetic particles with dT₁₄ oligomers covalently bound thereto. Another Target Capture Reagent may comprise HEPES, lithium hydroxide, LLS, Succinic Acid, with dT₁₄ oligomers covalently bound. Other formulations of Target Capture Reagent may function equally as well.

“Amplification Reagent” generally refers to a concentrated mixture of reaction components to facilitate amplification reactions. An Amplification Reagent will comprise a number of different reagents at various concentrations depending on factors such as for example amplification type (PCR, TMA, etc.), target nucleic acids (GC content), and the like. Primers may be added to the amplification reagent or added to amplification reactions separate from the amplification reagent. Enzymes in an amplification reagent can include one or more of Moloney Murine Leukemia Virus Reverse Transcriptase (MMLV-RT) and bacteriophage T7 RNA polymerase for which units are functionally defined as: 1 U of MMLV-RT incorporates 1 nmol of dTTP in 10 min at 37° C. using 200-400 micromolar oligo dT-primed poly(A) as template, and 1 U of T7 RNA polymerase incorporates 1 nmol of ATP into RNA in 1 hr at 37° C. using a DNA template containing a T7 promoter.

“Probe Reagent” generally refers to a solution containing one or more labeled detection probes.

TABLE 1
Oligomers of the SARS-CoV-2 assay. Lower case = methoxy RNA; Upper case = DNA

| SEQ ID NOs | Sequence (5′ to 3′) | Class |
|---|---|---|
| SEQ ID NO: 1 | AATTTAATACGACTCACTATAGGG | T7 Promote Primer (first |
| | AGAGTTCAATCTGTCAAGCAGCA | amplification oligomer) |
| | GCA | |
| SEQ ID NO: 3 | AATTTAATACGACTCACTATAGGG | T7 Promote Primer (first |
| | AGAGTTGTTGGCCTTTACCAGAC | amplification oligomer) |
| | AT | |
| SEQ ID NO: 5 | GAACTTCTCCTGCTAGAATG | Non-T7 Primer (second |
| | | amplification oligomer) |
| SEQ ID NO: 6 | GTAGGGGAACTTCTCCTGCTAG | Non-T7 Primer (second |
| | | amplification oligomer) |
| SEQ ID NO: 13 | uggcggugaugcugcucT | Detection Probe oligomer |
| SEQ ID NO: 8 | cucugcucccuucugcguagTTTAAAAA | Target capture oligo 348828 |
| | AAAAAAAAAAAAAAAAAAAAAAAA | |
| | A | |
| SEQ ID NO: 10 | gaugaggaacgagaagaggcTTTAAAAA | Target capture oligo 348829 |
| | AAAAAAAAAAAAAAAAAAAAAAAA | |
| | A | |

### Example 1: SARS-CoV-2 Assay Limit of Detection of SARS-CoV-2 in Sensitivity Panels

Purpose

The purpose of this study was to evaluate the analytical sensitivity and limit of detection (LOD) of the SARS-CoV-2 (SCV2) Assay on the Procleix Panther System for detection of SCV2 RNA.

Materials and Methods

Analytical Sensitivity panels consisted of a SCV2 in vitro transcript (IVT) from Bio-Synthesis (Lewisville, Tex.), Armored RNA Quant SARS-CoV-2 Control from Asuragen (Austin, Tex.), and AccuPlex SARS-CoV-2 Positive Reference Material from SeraCare (Milford, Mass.).

The IVT, corresponding to the appropriate sequence from GenBank accession number MN908947, was synthesized by Bio-Synthesis and quantitated by UV absorbance at 260 nm. The IVT panels were serially diluted in a HEPES detergent.

The Armored RNA Quant SARS-CoV-2 Control contains the SARS-CoV-2 viral nucleocapsid (N) sequence region. The Armored RNA panels were serially diluted in K2EDTA plasma donors acquired from Innovative Research (Novi, Mich.) and pooled from 16 donors.

The AccuPlex SARS-CoV-2 Positive Reference Material from SeraCare consists of a protein-coated RNA mix including ORF1a, RdRp, Envelope and Nucleopcapsid regions; nucleotides 417-1899, 3094-3360, 13291-13560, 18577-19051, 25801-28200, 27952-29873, respectively. The AccuPlex panels were serially diluted in K2EDTA plasma donors acquired from Innovative Research (Novi, Mich.) and pooled from 16 donors and in Viral Transport Medium (VTM) (Hank's Balanced Salt Solution, Fetal Bovine Serum, Fungizone). The dilutions in VTM were further diluted 1:1 in a HEPES detergent containing solution.

Analytical sensitivity panel members, frozen in 5.5 mL single use aliquots, were thawed at room temperature immediately before use. Regression Analysis using the Probit function in SAS (Gompertz Model) System software, Version 9.4 (Cary, N.C.) was used to calculate the 95% and 50% probability of detection levels.

Results

The average Relative Light Unit (RLU) and percent coefficient of variation (% CV) values calculated for samples containing RNA are from reactive results only (signal/cut-off values 1.0). The average Flasher RLU and % CV were calculated for the negative panel.

Detection of in vitro Transcript: The average RLU value for the SARS-CoV-2 Assay at 100 copies/mL was 1,121,151 RLU and at 30 copies/mL the average RLU value was 1,121,996 RLU.

Detection of Armored RNA Quant SARS-CoV-2 Control: The average RLU value for the SARS-CoV-2 Assay at 100 copies/mL was 991,549 RLU and at 30 copies/mL the average RLU value was 949,043 RLU.

Detection of AccuPlex SARS-CoV-2 Positive Reference Material: The average RLU value for the SARS-CoV-2 Assay at 100 copies/mL was 1,057,173 RLU and at 30 copies/mL the average RLU value was 1,048,391 RLU.

Detection of the AccuPlex SARS-CoV-2 Positive Reference Material diluted in VTM: The average RLU value for the SARS-CoV-2 Assay at 200 copies/mL was 1,082,489 RLU and at 60 copies/mL the average RLU value was 1,061,625 RLU.

Probit Analysis showed the predicted 50% and 95% detection rates in copies/mL from the results obtained from testing. The 95% detection probability for the in vitro transcript diluted in a HEPES detergent containing solution in the SARS-CoV-2 Assay on Panther was 20.67 copies/mL with a 95% Fiducial Limit of 14.26 to 37.34 copies/mL. The 95% detection probability for the Armored RNA Quant SARS-CoV-2 Control diluted in plasma in the SARS-CoV-2 Assay on Panther was 31.79 copies/mL with a 95% Fiducial Limit of 21.90 to 56.82 copies/mL. The 95% detection probability for the AccuPlex SARS-CoV-2 Positive Reference Material diluted in plasma in the SARS-CoV-2 Assay on Panther was 17.90 copies/mL with a 95% Fiducial Limit of 12.32 to 29.75 copies/mL. The 95% detection probability for the AccuPlex SARS-CoV-2 Positive Reference Material diluted in VTM in the SARS-CoV-2 Assay on Panther was 37.83 copies/mL with a 95% Fiducial Limit of 28.45 to 56.08 copies/mL.

Conclusion

The analytical sensitivity study demonstrated sensitive detection of SCV2 IVT in a HEPES detergent containing solution, armored RNA in plasma, AccuPlex Material in plasma, and AccuPlex material in VTM. Probit analysis gave 95% detection at 20.67 copies/mL in the SARS-CoV-2 Assay on Panther for in vitro transcript diluted in a HEPES detergent containing solution. Probit analysis gave 95% detection at 31.79 copies/mL in the SARS-CoV-2 Assay on Panther Armored RNA Quant SARS-CoV-2 Control diluted in plasma. Probit analysis gave 95% detection at 17.90 copies/mL in the SARS-CoV-2 Assay on Panther AccuPlex SARS-CoV-2 Positive Reference Material diluted in plasma. Probit analysis gave 95% detection at 37.83 copies/mL in the SARS-CoV-2 Assay on Panther AccuPlex SARS-CoV-2 Positive Reference Material diluted in VTM.

### Example 2: Inclusivity and Cross-Reactivity by in Silico Analysis

Purpose

The purpose of this study was to determine the inclusivity and cross-reactivity of the SARS-CoV-2 Assay using in silico analysis of oligos designs.

Materials and Methods

The inclusivity was evaluated using in silico analysis of the assay primers and probes (Table 1) in relation to SARS-CoV-2 (SCV2) sequences available in the GISAID and Genbank databases. Primer and probe conservation with publicly available sequence encompassing intended target regions was assessed by direct comparison to multiple sequence alignments.

The cross reactivity analysis was conducted using BLAST search of the NCBI database for primers against common pathogens observed in respiratory specimens, and blood borne pathogens. The nucleotide collection consists of GenBank+EMBL+DDBJ+PDB+RefSeq sequences, but excludes EST, STS, GSS, WGS, TSA, patent sequences as well as phase 0, 1, and 2 HTGS sequences and sequences longer than 100 Mb. The database is non-redundant. Identical sequences have been merged into one entry, while preserving the accession, GI, title and taxonomy information for each entry. The search parameters automatically adjust for short input sequences and the expect threshold is 1000; 5) The match and mismatch scores are 1 and −3, respectively; 6) The penalty to create and extend a gap in alignment is 5 and 2 respectively.

Results

Inclusivity (Analytical Sensitivity):

The inclusivity was evaluated using in silico analysis of the assay primers and probes in relation to SARS-CoV-2 sequences available in the GISAID (about 1700 related sequences up to the end of March 2020). Primer and probe conservation with publicly available sequence encompassing intended target regions was assessed by direct comparison to multiple sequence alignments. Percentage of sequence with 100% match is summarized in the Table 2.

TABLE 2
Percentage of 100% match for each oligo in the assay

| | | Number of 100% | |
|---|---|---|---|
| | | match/Total | Percentage of |
| SEQ ID NOs | Oligo | Sequence | 100% match |
| SEQ ID NO: 8 | Capture Oligos 1 | 1714/1715 | 99.9% |
| SEQ ID NO: 10 | Capture Oligos 2 | 1707/1715 | 99.5% |
| SEQ ID NO: 5 | Non-T7 Primer 1 | 1713/1715 | 99.9% |
| SEQ ID NO: 6 | Non-T7 Primer 2 | 1484/1715 | 86.5% |
| SEQ ID NO: 1 | T7 Primer 1 | 1715/1715 | 100.0% |
| SEQ ID NO: 3 | T7 Primer 2 | 1714/1715 | 99.9% |
| SEQ ID NO: 13 | Probe 1 | 1710/1715 | 99.7% |
| SEQ ID NO: 13 | Probe 2 | 1710/1715 | 99.7% |

The strategy of redundant primer for each capture probe oligomer, forward primer (T7-primer amplification oligomer), detection probe oligomer, and reverse primer (non-T7 amplification oligomer) mitigates the risk of mutations.

Meanwhile, alternative base with high affinity is used for target capture oligonucleotides and probes to create oligonucleotide backbones with enhanced hybridization properties. This type of oligonucleotide is able to detect minor viral variants without loss in sensitivity or specificity.

Based on this analysis and strategies applied in Procleix products and this assay, false negative results are not likely to occur with the oligonucleotides included in this system.

Cross-Reactivity (Analytical Specificity)

An in silico cross reactivity analysis was conducted using BLAST search of the NCBI database for primers against organism requested by the FDA EUA pre-submission process (in silico) guidelines (listed in Table 3) and other blood borne pathogens. The BLAST searches did not reveal any cross-reactivity with the exception of SARS coronavirus, which is in the same subgenus (Sarbecovirus) as SARS-CoV-2. Comparing to the SARS coronavirus (NC_004718) genome sequence, there are 90% homology to NT7 primers, and 90% homology to T7 primers, and 72% homology to probes. Therefore, cross-reactivity with SARS coronavirus is unlikely.

To evaluate the cross-reactivity with SARS coronavirus and other related Sarbecovirus, purified nucleic acid from those organisms was spiked into buffer matrix and tested using the SARS-CoV-2 assay. No cross-reactivity was observed for those Sarbecoviruse by preliminary testing.

TABLE 3
Microorganisms for cross-reactivity

| High priority pathogens from | High priority organisms | |
|---|---|---|
| the same genetic family | likely in circulating areas | Other Pathogens |
| Human coronavirus 229E | Adenovirus | CMV + G2: G21 |
| Human coronavirus OC43 | Human Metapneumovirus | EBV |
| Human coronavirus HKU1 | Parainfluenza virus 1-4 | HAV |
| Human coronavirus NL63 | Influenza A & B | HTLV 1 |
| SARS-coronavirus | Enterovirus | HTLV 2 |
| MERS-coronavirus | Respiratory syncytial virus | Parvovirus B19 |
| | Rhinovirus | rubella |
| | Chlamydia pneumoniae | Chikungunya virus |
| | Haemophilus influenzae | dengue 1 |
| | Legionella pneumophila | dengue 2 |
| | Mycobacterium tuberculosis | dengue 3 |
| | Streptococcus pneumoniae | dengue 4 |
| | Streptococcus pyogenes | WNV |
| | Bordetella pertussis | ZIKA virus |
| | Mycoplasma pneumoniae | HIV-1 |
| | Pneumocystis jirovecii (PJP) | HIV-2 |
| | Candida albicans | HCV |
| | Pseudomonas aeruginosa | HBV |
| | Staphylococcus epidermis | HEV |
| | Staphylococcus salivarius | |

Complementary Wet Testing of Pathogens from Same Genetic Family

Genomic RNA purified from some pathogens from same genetic family were spiked in the buffer solution, and tested using the SARS-CoV-2 assay on Panther instrument. The result is summarized in the Table 4. No cross reactivity was observed by the preliminary wet testing.

TABLE 4
Cross-reactivity with closely related coronaviruses

| | | RNA | | |
|---|---|---|---|---|
| Pathogen | PN | concentration | | Result |
| Human coronavirus OC43 | VR-1558DQ | ≥1 × 1e5 | c/mL | No Cross Reacitvity |
| Human coronavirus HKU1 | VR-3262SD | 1e5-1e6 | c/mL | No Cross Reacitvity |
| Human coronavirus NL63 | ATCC 3263SD | 1e5-1e6 | c/mL | No Cross Reacitvity |
| Human coronavirus 229E | ATCC VR-740DQ | ≥1 × 1e5 | c/mL | No Cross Reacitvity |
| SARS-CoV Frankfurt 1 | SARS-Cov1e4 | 1e5 | c/mL | No Cross Reacitvity |
| MERS-CoV RNA | VR-3248SD | 1e5-1e6 | c/mL | No Cross Reacitvity |

Conclusions

The in silico analysis of inclusivity of the SARS-CoV-2 assay design and the strategies applied in the assay (redundant primer and higher affinity oligonucleotides) revealed sufficient analytical sensitivity. False negative results are not likely to occur with the assay designs included in this assay.

The in silico analysis of cross-reactivity and the complementary wet testing with closely related coronavirus revealed sufficient analytical specificity. False positive results are not likely to occur with the assay designs included in this assay.

### Example 3: SARS-CoV-2 Assay Specificity in Normal Blood Donor Plasma Specimens

Purpose

The purpose of this study was to determine the specificity of the SARS-CoV-2 Assay on the Procleix Panther System. Normal blood donor plasma and serum specimens were tested.

Materials and Methods

Specimens from negative normal blood donors were obtained by Grifols Diagnostic Solutions Inc. from Seracare (Milford, Mass.) and ProMedDx (Norton, Mass.) and Creative Testing Solutions (CTS) in Charlotte, N.C. All specimens were pre-screened by the blood center from which the vendor obtained them, using licensed serological and nucleic acid test (NAT) assays, according to the blood center's standard protocol. Frozen specimens were thawed at room temperature the day of testing.

Results are expressed as signal to cutoff (S/CO) values. A sample is considered “Reactive” (positive) if the analyte S/CO is greater than or equal to 1.0. A sample is considered “Non-Reactive” (negative) if the analyte S/CO is <1.0 and the Internal Control (IC) S/CO>1.0. For any samples where both the analyte S/CO and the IC S/CO are <1.0, the sample is considered “Invalid”. Analyte and IC cutoff values are determined by the assay software based on the negative and positive calibrators included in each run.

True Negatives (TN) were defined as all negative specimens that gave a valid Non-Reactive result in the SARS-CoV-2 Assay. Initial Reactive (IR) samples were retested. False Positives (FP) were defined as specimens that were IR in the SARS-CoV-2 Assay but were not Repeat Reactive (RR) in any subsequent testing. True Positives (TP) were defined as specimens that were RR in the SARS-CoV-2 Assay. Specificity was calculated as follows:

Specificity=100×[#TN/(#TN+#FP)]

The 95% confidence interval (CI) was calculated using the Score Method (SAS Version 9.4).

Results

A total of 2332 normal blood donor specimens were tested with the SARS-CoV-2 Assay. The initial invalid rate due to assay chemistry errors was 0.04% (1/2407) for the SARS-CoV-2 Assay. The overall specificity was 99.7% (2326/2332) for the SARS-CoV-2 Assay with a lower 95% confidence interval of 99.4%.

### Example 4: Detection of a SARS-CoV-2 Nucleocapsid (N) Region

Purpose

The purpose of this study was to evaluate reactivity of the SARS-CoV-2 assay using different amplification oligomers directed to the nucleocapsid (N) gene region thereof. The experiment setup is shown in the following Table 5 and the sequences of capture and detection probes and amplification oligomers used are shown in Table 6:

TABLE 5
Experiment setup

| Exp. | Capture probe | Amplification oligomer | Detection probe |
|---|---|---|---|
| Amp | CVTC01 & 2 | CVNT02 & 3 CVT7A04 | CVP05 & 6 |
| 01 | | & 01 | |
| Amp | CVTC01 & 2 | CVNT02 & 3 CVT7A04 | CVP05 & 6 |
| 02 | | & 06 | |
| Amp | CVTC01 & 2 | CVNT01 & 3 CVT7A04 | CVP05 & 6 |
| 03 | | & 01 | |

TABLE 6
Sequences and SEQ ID NO. of capture and detection
probes, and amplification oligomers.

| Oligo | | SEQ ID |
|---|---|---|
| Name | Sequence (5′-3′) | NO: |
| CVP06 | UGGCGGUGAUGCU(2MeAE)GCUCT | 13 |
| CVP05 | UGGCGGUGAU(2MeAE)GCUGCUCT | 13 |
| CVTC02 | GAUGAGGAACGAGAAGAGGCTTTAAAAA | 10 |
| | AAAAAAAAAAAAAAAAAAAAAAAAA | |
| CVTC01 | CUCUGCUCCCUUCUGCGUAGTTTAAAAA | 8 |
| | AAAAAAAAAAAAAAAAAAAAAAAAA | |
| CVNT03 | GTAGGGGAACTTCTCCTGCTAG | 6 |
| CVNT02 | GAACTTCTCCTGCTAGAATG | 5 |
| CVNT01 | AACTTCTCCTGCTAGAATGGCTG | 44 |
| CVT7A06 | AATTTAATACGACTCACTATAGGGAGAGT | 22 |
| | TGTTGGCCTTTACCAGAC | |
| CVT7A04 | AATTTAATACGACTCACTATAGGGAGAGT | 3 |
| | TGTTGGCCTTTACCAGACAT | |
| CVT7A01 | AATTTAATACGACTCACTATAGGGAGAGT | 1 |
| | TCAATCTGTCAAGCAGCAGCA | |

Materials and Methods

Initial primer screening was performed using Transcription-Mediated Amplification (TMA) on the manual Procleix system using SARS-CoV-2 in vitro transcript (IVT). An assay rack consisted of 10 rows of Ten-tube units (TTUs). Four hundred microliters (400 μL) of Target Capture Reagent (suitable reagents outlined above) and 5 picomoles of each Target capture oligonucleotide were added to the appropriate tubes on the rack such that each combination of amplification oligomers were tested with 10 replicates of negative control and 10 replicates of SARS-CoV-2 IVT at 001, 005, 020, and 100 copies/reaction. Sample/TCR combinations were vortexed for 20 seconds, incubated for 20 minutes at 60±1° C., incubated at room temperature for 15 minutes and placed on the Target Capture Station (TCS) test tube bay for 10 minutes. Fluid was removed from the test tubes, and 1 mL of wash solution was added. Tubes were vortexed for 20 seconds and placed back on the TCS test tube bay for 5 minutes. The step aspiration/wash/hold were repeated. Final wash buffer was aspirated, and tubes were removed for the TCS test tube bay. Seventy five microliters (75 μL) of Amplification Reagent and 5 picomoles of each T7 promoter provider oligonucleotide and non-T7 primer oligonucleotide were added to the appropriate tubes, 200 μL of oil was added to each tube and then the rack was covered with sealing cards and vortexed for a minimum of 20 seconds.

The rack was then incubated in a water bath at 60±1° C. for 10±1 minutes followed by incubation in a 41.5±1° C. water bath for 20 minutes. While the rack remained in the water bath, the sealing cards were removed and 25 μL of commercially available Procleix enzyme reagent (Grifols Diagnostic Solutions Inc.) was added to each reaction tube and then covered again with sealing cards. The rack was gently shaken to mix and then covered again with sealing cards and incubated for another 60±5 minutes in the 41.5±1° C. water bath.

After incubation completed, the rack was transferred to the hybridization protection assay (HPA) area where the sealing cards were removed. 100 μL of Probe reagent consisting of an Acridinium-Ester (AE) labeled probe added at a total desired concentration of at least 2.5e6 Relative Light Units (RLU) per reaction to a Hybridization reagent. Probe reagent was then added to the appropriate reaction tubes. The tubes were covered with sealing cards and the rack was vortexed for a minimum of 20 seconds after which the rack was incubated in a water bath at 61±2° C. for 15±1 minutes.

The rack was removed from the water bath, the sealing cards removed, and 250 μL of commercially available Procleix Selection reagent (Grifols Diagnostic Solutions Inc.) was added to each tube. The tubes were covered with sealing cards and vortexed for a minimum of 20 seconds and then returned to the 61±2° C. water bath and incubated for 10±1 minutes. After incubation the rack was allowed to cool in a 23±4° C. water bath for a minimum of 10 minutes.

For detection the TTUs are removed from the rack and loaded on to the automated Leader instrument for subsequent light off using commercially available Procleix Auto Detect 1 and 2 reagents (Grifols Diagnostic Solutions Inc.) and the results were exported for analysis of the signal in Relative Light Units (RLU).

Results

Percent reactivity (%) was measured based of the number of replicates with RLUs above the background RLUs of the negative control for different number of DNA copies (1, 5, 20, and 100 copies). Results are shown in Table 7.

TABLE 7
Reactivity (%) of each of the experiments

| | % Reactivity | | |
|---|---|---|---|
| Target | Amp 01 | Amp 02 | Amp 03 |
| Negative control | 0% | 0% | 10% |
| nCoV 001 copies | 40% | 20% | 30% |
| nCoV 005 copies | 60% | 60% | 80% |
| nCoV 020 copies | 100% | 100% | 100% |
| nCoV 100 copies | 100% | 100% | 100% |

In this experiment, all combinations showed good reactivity with 100% detection at 020 copies/reaction and above. Amp03 showed 10% reactivity 10% in the negative control.

### Example 5: Detection of a SARS-CoV-2 Spike (S) Region

Purpose

The purpose of this study was to evaluate reactivity of the SARS-CoV-2 assay using different amplification oligomers directed to the Spike (S) gene region thereof. The experiment setup is shown in the following Table 8 and the sequences of capture and detection probes and amplification oligomers used are shown in Table 9:

TABLE 8
Experiment setup

| System | Capture probe | Amplification oligomer | Detection probe |
|---|---|---|---|
| A | CVSTC01 & 3 | CVNT7S01 & 3_CVT7S04 | CVSP01 & 3 |
| | | & 05 | |
| B | CVSTC01 & 3 | CVNT7S01 & 3_CVT7S03 | CVSP01 & 3 |
| | | & 05 | |
| C | CVSTC01 & 3 | CVNT7S02 & 3_CVT7S04 | CVSP01 & 3 |
| | | & 05 | |

TABLE 9
Sequences and SEQ ID NO. of capture and detection
probes, and amplification oligomers.

| | | SEQ ID |
|---|---|---|
| Oligo Name | Sequence (5′-3′) | NO: |
| CVSP03 | GACCCAGUCC(2MeAE)CUACUUAT | 36 |
| CVSP01 | GACCCA(2MeAE)GUCCCUACUUAT | 36 |
| CVSTCS03 | GAACUCACUUUCCAUCCAACTTTAAAA | 34 |
| | AAAAAAAAAAAAAAAAAAAAAAAAAA | |
| CVSTCS01 | UGUUAGACUUCUCAGUGGAAGCTTTA | 32 |
| | AAAAAAAAAAAAAAAAAAAAAAAAAAAA | |
| | A | |
| CVT7S05 | AATTTAATACGACTCACTATAGGGAGA | 28 |
| | TTGAAATTCACAGACTTTAATAACA | |
| CVT7S04 | AATTTAATACGACTCACTATAGGGAGA | 26 |
| | CTTTAATAACAACATTAG | |
| CVNT7S01 | GTACTACTTTAGATTCGAAG | 30 |
| CVNT7S03 | GATTTTTGGTACTACTTTAG | 31 |
| CVT7S03 | AATTTAATACGACTCACTATAGGGAGA | 45 |
| | CTTTAATAACAACATTAGT | |
| CVNT7S02 | GGATTTTTGGTACTACTTTAGA | 46 |

Materials and Methods

The Materials and Methods was identical to the ones described for Example 4.

Results

As shown in Table 10, all the systems showed 100% reactivity at 020 copies/reaction and above. System A performed best with 50% reactivity down to 001 copy/reaction and 1 to 3 percent of Coefficient of Variability (% CV) on the mean RLU values at 005 copies/reaction and above, showing a robust and consistent signal for the detection of low SARS-CoV-2 RNA detection.

TABLE 10
Results of each of the combinations tested
in the experiment of Example 5.

| Sample | SARS-CoV-2 S-gene System A | | | | | |
|---|---|---|---|---|---|---|
| (copies/reaction) | N | #R | % R | Mean RLUs | SD | % CV |
| 100 | 10 | 10 | 100 | 1.700.168 | 55.805 | 3% |
| 020 | 10 | 10 | 100 | 1.675.698 | 51.248 | 3% |
| 005 | 10 | 5 | 50 | 1.714.408 | 23.740 | 1% |
| 001 | 10 | 5 | 50 | 1.583.087 | 290.820 | 18% |
| Negative control | 10 | 0 | 0 | 152.628 | 59.121 | 0% |
| Sample | SARS-CoV-2 S-gene System B | | | | | |
| (copies/reaction) | N | #R | % R | Mean RLUs | SD | % CV |
| 100 | 10 | 10 | 100 | 1.721.949 | 25.396 | 1% |
| 020 | 10 | 10 | 100 | 1.723.664 | 27.178 | 2% |
| 005 | 10 | 4 | 40 | 1.083.932 | 710.660 | 66% |
| 001 | 10 | 1 | 10 | 1.701.778 | NA | NA |
| Negative control | 10 | 0 | 0 | 174.177 | 6.269 | 4% |
| Sample | SARS-CoV-2 S-gene System C | | | | | |
| (copies/reaction) | N | #R | % R | Mean RLUs | SD | % CV |
| 100 | 10 | 10 | 100 | 1.713.591 | 29.107 | 2% |
| 020 | 10 | 10 | 100 | 1.742.358 | 78.119 | 4% |
| 005 | 10 | 1 | 10 | 1.758.916 | NA | NA |
| 001 | 8 | 1 | 12.5 | 1.715.580 | NA | NA |
| Negative control | 10 | 0 | 0 | 171.027 | 9.011 | 5% |

N: number valid,
#: number,
R: Reactive,
RLUs: Relative Light Units,
SD: Standard Deviation,
CV: Coefficient of variation

SEQUENCES

| SEQ ID NOs | Sequence (5′ to 3′) |
|---|---|
| SEQ ID NO: 1 | AATTTAATACGACTCACTATAGGGAGAGTTCAATCTGTCAAGCAGC |
| | AGCA |
| SEQ ID NO: 2 | GTTCAATCTGTCAAGCAGCAGCA |
| SEQ ID NO: 3 | AATTTAATACGACTCACTATAGGGAGAGTTGTTGGCCTTTACCAGA |
| | CAT |
| SEQ ID NO: 4 | GTTGTTGGCCTTTACCAGACAT |
| SEQ ID NO: 5 | GAACTTCTCCTGCTAGAATG |
| SEQ ID NO: 6 | GTAGGGGAACTTCTCCTGCTAG |
| SEQ ID NO: 7 | AATTTAATACGACTCACTATAGGGAGA |
| SEQ ID NO: 8 | cucugcucccuucugcguagTTTAAAAAAAAAAAAAAAAAAAAAAAAAAAA |
| | AA |
| SEQ ID NO: 9 | Cucugcucccuucugcguag |
| SEQ ID NO: 10 | gaugaggaacgagaagaggcTTTAAAAAAAAAAAAAAAAAAAAAAAAAAAA |
| | AA |
| SEQ ID NO: 11 | Gaugaggaacgagaagaggc |
| SEQ ID NO: 12 | TTTAAAAAAAAAAAAAAAAAAAAAAAAAAAAAA |
| SEQ ID NO: 13 | uggcggugaugcugcucT |
| SEQ ID NO: 14 | ccacaagcuuagaagauagagagG |
| SEQ ID NO: 15 | CTATCTTCTAAGCTTG |
| SEQ ID NO: 16 | CTTCTACGCAGAAGGGAGCAGAGGCGGCAGTCAAGCCTCTTCTC |
| | GTTCCTCATCACGTAGTCGCAACAGTTCAAGAAATTCAACTCCAGG |
| | CAGCAGTAGGGGAACTTCTCCTGCTAGAATGGCTGGCAATGGCG |
| | GTGATGCTGCTCTTGCTTTGCTGCTGCTTGACAGATTGAACCAGC |
| | TTGAGAGCAAAATGTCTGGTAAAGGCCAACAACAACAAGGCCAAA |
| | CTGTCACTAA |
| SEQ ID NO: 17 | AATGGCTGGCAATGGCGGTGATGCTGCTCTTGCTTTGCTGCTGCT |
| | TGACAGATTGAACCAGCTTGAGAGCAAA |
| SEQ ID NO: 18 | GCTGGCAATGGCGGTGATGCTGCTCTTGCTTTGCTGCTGCTTGAC |
| | AGATTGAACCAGCTTGAGAGCAAA |
| SEQ ID NO: 19 | AATGGCTGGCAATGGCGGTGATGCTGCTCTT |
| SEQ ID NO: 20 | GCTGGCAATGGCGGTGATGCTGCTCTT |
| SEQ ID NO: 21 | AATGGCGGTGATGCTGCTCTT |
| SEQ ID NO: 22 | AATTTAATACGACTCACTATAGGGAGAGTTGTTGGCCTTTACCAGA |
| | C |
| SEQ ID NO: 23 | GTTGTTGGCCTTTACCAGAC |
| SEQ ID NO: 24 | GCTGGCAATGGCGGTGATGCTGCTCTTGCTTTGCTGCTGCTTGAC |
| | AGATTGAACCAGCTTGAGAGCAAAAT |
| SEQ ID NO: 25 | AATGGCTGGCAATGGCGGTGATGCTGCTCTTGCTTTGCTGCTGCT |
| | TGACAGATTGAACCAGCTTGAGAGCAAAAT |
| SEQ ID NO: 26 | AATTTAATACGACTCACTATAGGGAGActttaataacaacattag |
| SEQ ID NO: 27 | ctttaataacaacattag |
| SEQ ID NO: 28 | AATTTAATACGACTCACTATAGGGAGAttgaaattcacagactttaataaca |
| SEQ ID NO: 29 | ttgaaattcacagactttaataaca |
| SEQ ID NO: 30 | GTACTACTTTAGATTCGAAG |
| SEQ ID NO: 31 | GATTTTTGGTACTACTTTAG |
| SEQ ID NO: 32 | uguuagacuucucaguggaagcTTTAAAAAAAAAAAAAAAAAAAAAAAAAA |
| | AAAA |
| SEQ ID NO: 33 | uguuagacuucucaguggaagc |
| SEQ ID NO: 34 | gaacucacuuuccauccaacTTTAAAAAAAAAAAAAAAAAAAAAAAAAAAA |
| | AA |
| SEQ ID NO: 35 | gaacucacuuuccauccaac |
| SEQ ID NO: 36 | gacccagucccuacuuaT |
| SEQ ID NO: 37 | AAGTAGGGACTGGGTC |
| SEQ ID NO: 38 | AgAgCaGcAuCaCc |
| SEQ ID NO: 39 | TATACATGTCTCTGGGACCAATGGTACTAAGAGGTTTGATAACCCT |
| | GTCCTACCATTTAATGATGGTGTTTATTTTGCTTCCACTGAGAAGT |
| | CTAACATAATAAGAGGCTGGATTTTTGGTACTACTTTAGATTCGAA |
| | GACCCAGTCCCTACTTATTGTTAATAACGCTACTAATGTTGTTATTA |
| | AAGTCTGTGAATTTCAATTTTGTAATGATCCATTTTTGGGTGTTTAT |
| | TACCACAAAAACAACAAAAGTTGGATGGAAAGTGAGTTCAGAGTTT |
| | ATTCTAGTGCGAATAATTGCACTTTTGAATATGT |
| SEQ ID NO: 40 | ACCCAGTCCCTACTTATTGTTAATAACGCTA |
| SEQ ID NO: 41 | ATTCGAAGACCCAGTCCCTACTTATTGTTAATAACGCTA |
| SEQ ID NO: 42 | ACCCAGTCCCTACTTATTGTTAATAACGCTACTAATGT |
| SEQ ID NO: 43 | ATTCGAAGACCCAGTCCCTACTTATTGTTAATAACGCTACTAATGT |
| SEQ ID NO: 44 | AACTTCTCCTGCTAGAATGGCTG |
| SEQ ID NO: 45 | AATTTAATACGACTCACTATAGGGAGACTTTAATAACAACATTAGT |
| SEQ ID NO: 46 | GGATTTTTGGTACTACTTTAGA |

Note that the amplicon and partial amplicon sequences are illustrated herein as DNA, however, the skilled person understands that amplification products generated during TMA reactions are either RNA or DNA, depending upon the stage in the amplification cycle. DNA designation is provided herein only for convenience, and not limitation.

1. A method for detecting SARS-CoV-2 nucleic acid in a sample, said method comprising:
(1) contacting a sample suspected of containing SARS-CoV-2 nucleic acid, with at least two amplification oligomers for amplifying at least one target region of a SARS-CoV-2 target nucleic acid, wherein said at least two amplification oligomers comprise:
(I) a first and a second amplification oligomers for amplifying a first target region of a SARS-CoV-2 nucleic acid, wherein
(a) the first amplification oligomer comprises a first target-hybridizing sequence comprising or consisting of a sequence selected from the group consisting of SEQ ID NO:2, SEQ ID NO:4 and SEQ ID NO:23; and
(b) the second amplification oligomer comprises a second target-hybridizing sequence comprising or consisting of a sequence selected from the group consisting of SEQ ID NO:5, and SEQ ID NO:6;
and/or
(II) a first and a second amplification oligomers for amplifying a second target region of a SARS-CoV-2 nucleic acid, wherein
(a) the first amplification oligomer comprises a first target-hybridizing sequence comprising or consisting of a sequence selected from the group consisting of SEQ ID NO:27 and SEQ ID NO:29; and
(b) the second amplification oligomer comprises a second target-hybridizing sequence comprising or consisting of a sequence selected from the group consisting of SEQ ID NO:30 and SEQ ID NO:31;
(2) performing an in vitro nucleic acid amplification reaction, wherein any SARS-CoV-2 target nucleic acid present in said sample is used as a template for generating an amplification product; and
(3) detecting the presence or absence of the amplification product, thereby indicating the presence or absence of SARS-CoV-2 target nucleic acid in said sample.

2-5. (canceled)

6. The method of claim 1, wherein the first amplification oligomer for amplifying the first and/or the second target region is a promoter primer or promoter provider further comprising a promoter sequence located 5′ to the first target-hybridizing sequence.

7. The method of claim 6, wherein the promoter sequence is a T7 promoter sequence.

8. (canceled)

9. The method of claim 1, wherein the first and second target-hybridizing sequences of the first and second amplification oligomers for amplifying the first target region and/or the second target region, respectively comprise or consist of the nucleotide sequences of:
i) for amplifying the first target region:
(a) SEQ ID NO:2, SEQ ID NO:4 or SEQ ID NO:23 and SEQ ID NO:5; or
(b) SEQ ID NO:2 SEQ ID NO:4 or SEQ ID NO:23 and SEQ ID NO:6; or
(c) SEQ ID NO:2 and SEQ ID NO:5 or SEQ ID NO:6; or
(d) SEQ ID NO:4 and SEQ ID NO:5 or SEQ ID NO:6; or
(e) SEQ ID NO:23 and SEQ ID NO:5 or SEQ ID NO:6; or
(f) SEQ ID NO:2 and SEQ ID NO:5; or
(g) SEQ ID NO:2 and SEQ ID NO:6; or
(h) SEQ ID NO:4 and SEQ ID NO:5; or
(i) SEQ ID NO:4 and SEQ ID NO:6, or
(j) SEQ ID NO:23 and SEQ ID NO:5; or
(k) SEQ ID NO:23 and SEQ ID NO:6;
ii) for amplifying the second target region:
(a) SEQ ID NO:27 or SEQ ID NO:29 and SEQ ID NO:30, or
(b) SEQ ID NO:27 or SEQ ID NO:29 and SEQ ID NO:31, or
(c) SEQ ID NO:27 and SEQ ID NO:30 or SEQ ID NO:31, or
(d) SEQ ID NO:29 and SEQ ID NO:30 or SEQ ID NO:31, or
(e) SEQ ID NO:27 and SEQ ID NO:30, or
(f) SEQ ID NO:27 and SEQ ID NO:31, or
(g) SEQ ID NO:29 and SEQ ID NO:30, or
(h) SEQ ID NO:29 and SEQ ID NO:31.

10. (canceled)

11. The method of claim 1, wherein the at least two amplification oligomers for amplifying the first target region and/or the second target region comprise redundant first amplification oligomers and/or redundant second amplification oligomers.

12. The method of claim 11, wherein the first and second target-hybridizing sequences of the redundant first and/or second amplification oligomers for amplifying the first target region and/or the second target region, respectively comprise or consist of the nucleotide sequences of:
i) for amplifying the first target region:
(a) SEQ ID NO:2 and SEQ ID NO:4 and SEQ ID NO:5; or
(b) SEQ ID NO:2 and SEQ ID NO:4 and SEQ ID NO:6; or
(c) SEQ ID NO:2 and SEQ ID NO:5 and SEQ ID NO:6; or
(d) SEQ ID NO:4 and SEQ ID NO:5 and SEQ ID NO:6, or
(e) SEQ ID NO:2 and SEQ ID NO:4 and SEQ ID NO:5 and SEQ ID NO:6, or
(f) SEQ ID NO:2 and SEQ ID NO:23 and SEQ ID NO:5; or
(g) SEQ ID NO:2 and SEQ ID NO:23 and SEQ ID NO:6; or;
(h) SEQ ID NO:23 and SEQ ID NO:5 and SEQ ID NO:6, or
(i) SEQ ID NO:2 and SEQ ID NO:23 and SEQ ID NO:5 and SEQ ID NO:6, or
(j) SEQ ID NO:4 and SEQ ID NO:23 and SEQ ID NO:5; or
(k) SEQ ID NO:4 and SEQ ID NO:23 and SEQ ID NO:6; or;
(l) SEQ ID NO:4 and SEQ ID NO:23 and SEQ ID NO:5 and SEQ ID NO:6;
ii) for amplifying the second target region
(a) SEQ ID NO:27 and SEQ ID NO:29 and SEQ ID NO:30, or
(b) SEQ ID NO:27 and SEQ ID NO:29 and SEQ ID NO:31, or
(c) SEQ ID NO:27 and SEQ ID NO:30 and SEQ ID NO:31, or
(d) SEQ ID NO:29 and SEQ ID NO:30 and SEQ ID NO:31, or
(e) SEQ ID NO:27 and SEQ ID NO:29 and SEQ ID NO:30 and SEQ ID NO:31.

13-14. (canceled)

15. The method of claim 12, wherein the redundant first and/or second amplification oligomers for amplifying the first and the second target regions respectively comprise or consist of the nucleotide sequences of:
(a) SEQ ID NO:2, SEQ ID NO:4, SEQ ID NO:5, SEQ ID NO:6,_SEQ ID NO:27, SEQ ID NO:29, SEQ ID NO:30 and SEQ ID NO:31, or
(b) SEQ ID NO:2, SEQ ID NO:23, SEQ ID NO:5. SEQ ID NO:6, SEQ ID N:27, SEQ ID NO:29, SEQ ID NO:30 and SEQ ID NO:31, or
(c) SEQ ID N:4, SEQ ID NO:23, SEQ ID NO:5, SEQ ID NO:6, SEQ ID NO:27, SEQ ID NO:29, SEQ ID NO:30 and SEQ ID NO:31.

16. The method of claim 1, further comprising purifying the target nucleic acid from other components in the sample before step (1).

17. The method of claim 16, wherein the purifying step comprises contacting the sample with at least one capture probe oligomer comprising a target-hybridizing sequence covalently attached to a sequence or moiety that binds to an immobilized probe, wherein said target-hybridizing sequence comprises or consists of a sequence selected from the group consisting of SEQ ID NO:9, and SEQ ID NO:11 or from the group consisting of SEQ ID NO:33, and SEQ ID NO:35.

18-20. (canceled)

21. The method of claim 1, wherein the detecting step (3) comprises contacting said in vitro nucleic acid amplification reaction with at least one detection probe oligomer hybridizes to the amplification product under conditions whereby the presence or absence of the amplification product is determined, thereby indicating the presence or absence of SARS-CoV-2 in said sample.

22. The method of claim 21, wherein the at least one detection probe oligomer comprises a target-hybridizing sequence that is from about 14 to about 40 nucleotides in length and hybridizes to a target sequence comprising or consisting of
a) SEQ ID NO:13, the DNA equivalent of SEQ ID NO:13, the complement of SEQ ID NO:13, the DNA equivalent of the complement of SEQ ID NO:13, or the DNA/RNA chimeric of SEQ ID NO:13, or SEQ ID NO:25, the RNA equivalent of SEQ ID NO:25, the complement of SEQ ID NO:25, the RNA equivalent of the complement of SEQ ID NO:25, or the DNA/RNA chimeric thereof and/or;
b) SEQ ID NO:36, the DNA equivalent of SEQ ID NO: 36, the complement of SEQ ID NO: 36, the DNA equivalent of the complement of SEQ ID NO: 36, or the DNA/RNA chimeric of SEQ ID NO: 36, or SEQ ID NO:43, the RNA equivalent of SEQ ID NO:43, the complement of SEQ ID NO:43, the RNA equivalent of the complement of SEQ ID NO:43, or the DNA/RNA chimeric thereof.

23-30. (canceled)

31. The method of claim 29, wherein the at least two amplification oligomers for amplifying the first and/or the second target region comprise redundant first amplification oligomers and/or redundant second amplification oligomers and/or redundant detection probe oligomers.

32-33. (canceled)

34. The method of claim 21, wherein the detection probe oligomer comprises a label selected from the group consisting of a chemiluminescent label, a fluorescent label, quencher and a combination of one or more thereof.

35-36. (canceled)

37. The method of claim 1, wherein the amplification reaction at step (2) is an isothermal amplification reaction.

38. The method of claim 37, wherein the isothermal amplification reaction is a transcription-mediated amplification (TMA) reaction.

39. The method of claim 1, wherein the detection step (3) is a hybridization protection assay (HPA).

40. The method of claim 1, wherein the sample is a clinical sample, a blood sample, a plasma sample, or a serum sample.

41-119. (canceled)

120. A composition comprising:
a detection probe oligomer for specifically detecting a SARS-CoV-2 target nucleic acid in a sample, said detection probe oligomer comprising a target-hybridizing sequence that is from about 14 to about 40 nucleotides in length and is configured to specifically hybridize to a target sequence comprising or consisting of SEQ ID NO:13 or SEQ ID NO: 36, the DNA equivalent of SEQ ID NO:13 or SEQ ID NO: 36, the complement of SEQ ID NO:13 or SEQ ID NO: 36, the DNA equivalent of the complement of SEQ ID NO:13 or SEQ ID NO: 36, or the DNA/RNA chimeric of SEQ ID NO:13 or SEQ ID NO: 36, or SEQ ID NO:25 or SEQ ID NO: 43, the RNA equivalent of SEQ ID NO:25 or SEQ ID NO: 43, the complement of SEQ ID NO:25 or SEQ ID NO: 43, the RNA equivalent of the complement of SEQ ID NO:25 or SEQ ID NO: 43, or the DNA/RNA chimeric thereof.

121. The composition of claim 120, wherein the detection probe target-hybridizing sequence is contained in the sequence of SEQ ID NO:17 or SEQ ID NO: 41, and includes at least the sequence of SEQ ID NO:13 or SEQ ID NO: 36.

122-123. (canceled)

124. The composition of claim 120, wherein the detection probe oligomer comprises a label selected from the group consisting of a chemiluminescent label, a fluorescent label, quencher and a combination of one or more thereof.

125-135. (canceled)
